(19) United States
(12) Patent Application Publication
(10) Pub. No.: US 2012/0046528 A1
(43) Pub. Date: Feb. 23, 2012

(54) SYSTEM AND METHOD FOR DETECTING AND TREATING CARDIOVASCULAR DISEASE

(75) Inventors: Neal L. Eigler, Malibu, CA (US); James S. Whiting, Los Angeles, CA (US); Brian M. Mann, Edgartown, MA (US)

(73) Assignee: PACESETTER, INC., Sylmar, CA (US)

(21) Appl. No.: 12/857,914

(22) Filed: Aug. 17, 2010

(51) Int. Cl. A61B 5/02; A61B 5/00

(52) U.S. Cl. 600/301; 600/508

(57) ABSTRACT

A system for detecting and treating congestive heart failure includes an implantable module, such as a pacemaker, and a patient advisory module. The system is configured to measure thoracic impedance and to provide the patient with instructions in order to improve the accuracy of the thoracic impedance measurement as well as treating symptoms of congestive heart failure.

### FIELD OF THE INVENTION

This invention relates generally to systems and methods for detecting, diagnosing and treating cardiovascular disease in a medical patient, including instructions to the patient to improve the accuracy of its physiological measurements during and/or permit the patient to provide information that can be used to improve the accuracy of the physiological measurements.

### BACKGROUND OF THE INVENTION

The optimum management of patients with chronic diseases requires that therapy be adjusted in response to changes in the patient's condition. Ideally, these changes are measured by daily patient self-monitoring prior to the development of symptoms. Self-monitoring and self-administration of therapy forms a closed therapeutic loop, creating a dynamic management system for maintaining homeostasis. Such a system can, in the short term, benefit day-to-day symptoms and quality-of-life, and in the long term, prevent progressive deterioration and complications.

In some cases, timely administration of a single dose of a therapy can prevent serious acute changes in the patient's condition. One example of such a short-term disease management strategy is commonly used in patients with asthma. The patient acutely self-administers an inhaled bronchodilator when daily readings from a hand-held spirometer or flowmeter exceed a normal range. This has been effective for preventing or aborting acute asthmatic attacks that could lead to hospitalization or death

In another chronic disease, diabetes mellitus, current self-management strategies impact both the short and long term sequelae of the illness. Diabetic patients self-monitor blood glucose levels from one to three times daily and correspondingly adjust their self-administered injectable insulin or oral hypoglycemic medications according to their physician's prescription (known as a “sliding scale”). More “brittle” patients, usually those with juvenile-onset diabetes, may require more frequent monitoring (e.g., 4 to 6 times daily), and the readings may be used to adjust an external insulin pump to more precisely control glucose homeostasis. These frequent “parameter-driven” changes in diabetes management prevent hospitalization due to symptoms caused by under-treatment (e.g., hyperglycemia with increased hunger, thirst, urination, blurred vision), and over-treatment (e.g., hypoglycemia with sweating, palpitations, and weakness). Moreover, these aggressive management strategies have been shown to prevent or delay the onset of long-term complications, including blindness, kidney failure, and cardiovascular disease.

There are approximately 60 million people in the U.S. with risk factors for developing chronic cardiovascular diseases, including high blood pressure, diabetes, coronary artery disease, valvular heart disease, congenital heart disease, cardiomyopathy, and other disorders. Another 10 million patients have already suffered quantifiable structural heart damage but are presently asymptomatic. Still yet, there are 5 million patients with symptoms relating to underlying heart damage defining a clinical condition known as congestive heart failure (CHF). Although survival rates have improved, the mortality associated with CHF remains worse than many common cancers. The number of CHF patients is expected to grow to 10 million within the coming decade as the population ages and more people with damaged hearts are surviving.

CHF is a condition in which a patient's heart works less efficiently than it should, and a condition in which the heart fails to supply the body sufficiently with the oxygen-rich blood it requires, either during exercise or at rest. To compensate for this condition and to maintain blood flow (cardiac output), the body retains sodium and water such that there is a build-up of fluid hydrostatic pressure in the pulmonary blood vessels that drain the lungs. As this hydrostatic pressure overwhelms oncotic pressure and lymph flow, fluid shifts from the pulmonary veins into the pulmonary interstitial spaces, and eventually into the alveolar air spaces. This complication of CHF is called pulmonary edema, which can cause shortness of breath, hypoxemia, acidosis, respiratory arrest, and death. Although CHF is a chronic condition, the disease often requires acute hospital care. Patients are commonly admitted for acute pulmonary congestion accompanied by serious or severe shortness of breath. Acute care for congestive heart failure accounts for the use of more hospital days than any other cardiac diagnosis, and consumes in excess of 20 billion dollars in the United States annually.

Cardiac rhythm management devices such as pacemakers are an important tool in the treatment of cardiovascular diseases. Typically, an implantable pacemaker uses a minimum of two electrodes to stimulate tissue. At least one of these electrodes is in contact with the heart tissue to be stimulated, and is called a pacing electrode. The required second electrode need not be in contact with tissue being stimulated, in which case it is called an “indifferent” electrode. The indifferent electrode does not even have to be in the heart. Cardiac pacemakers in commercial use today all have the same basic configuration in which stimulating electrical pulses are produced by a pulse generator located outside the heart, typically in a subcutaneous pocket in the upper chest near one shoulder. The stimulating electrical pulses are applied to the electrodes via one or more electrical conductors within an insulated flexible cable, or “lead”, which is connected at its proximal end to the pulse generator. The distal end of the lead is placed within the heart at a desired pacing location, for example in the apex of the right ventricle. Some pacemaker leads, called “unipolar” leads, have only a pacing electrode, typically at the distal end of the lead. In this case, the required indifferent electrode may be provided by the metallic housing of the generator, or conceivably could be located on another lead. Commonly, unipolar pacemaker leads have a single conductor connecting the generator to a single pacing electrode located at its distal end. Bipolar pacemaker leads have two conductors, one connected to a pacing electrode located at or near the distal end of the lead, the other connected to an “indifferent” electrode, usually configured as a ring electrode, located on the lead some distance proximal to its distal end.

### SUMMARY OF THE INVENTION

Pressure within the left atrium of the heart is the precursor of fluid accumulation in the lungs, which results in signs and symptoms of acute CHF. Mean left atrial pressure in healthy individuals is normally less than or equal to twelve millimeters of mercury (mm Hg). Transudation of fluid into the pulmonary interstitial spaces can be expected to occur when the left atrial pressure is above about twenty-five mm Hg, or at somewhat more than about thirty mm Hg in some patients with chronic CHF. Pulmonary edema has been found to be predicted by reference to left atrial pressures and less well correlated with conditions in any other chamber of the heart.

Measurements of thoracic impedance have been experimentally shown to be inversely correlated with left atrial pressure. The use of thoracic impedance is premised on the theory that body fluids are the most electrically conductive material in the chest cavity. As fluid retention in the heart chambers and lung tissue increases with pulmonary edema, electrical resistance through the chest cavity decreases. Therefore, measurements of thoracic impedance can function as an indirect measure of left atrial pressure. One advantage of measuring thoracic impedance instead of or in addition to left atrial pressure is that there is no need to measure an atmospheric reference pressure. Another advantage of measuring thoracic impedance over measuring left atrial pressure directly with a pressure sensor is that thoracic impedance can be measured without implanting an additional sensor directly in the left atrium, but instead, can be measured using existing leads and the pacemaker housing.

Several embodiments of the present invention relate to systems and methods for detecting, diagnosing and treating cardiovascular disease in a medical patient using patient instructions from a patient interface-device interface to alter extrinsic factors to improve the accuracy and/or reproducibility of physiological measurements. In one embodiment, the invention comprises a method for treating cardiovascular disease (such as CHF) in a medical patient comprising determining a thoracic impedance and the patient's spatial orientation (such as the patient's location and/or position) and communicating the thoracic impedance and spatial orientation to a signal processing apparatus. The method further comprises operating the signal processing apparatus to generate a signal indicative of an appropriate therapeutic treatment to the patient, and communicating that signal to the patient.

In several embodiments, the patient's spatial orientation is determined by querying the patient. The patient can be asked to provide information regarding his or her spatial orientation (e.g., location, position, etc) over the phone, by email, or other communication means. In other embodiments, the patient's spatial orientation is determined without active patient participation. For example, spatial orientation can be determined by using one or more sensors configured to determine the patient's spatial orientation. The sensor comprises an accelerometer and/or a multiaxis tiltometer.

In several embodiments, instead of determining the patient's spatial orientation, the patient is signaled or otherwise instructed to assume a certain spatial orientation. For example, using the telephone, email, mechanical vibration, or another communication device, the patient is instructed to adopt a prone position, sit, or stand. In yet other embodiments, the patient is instructed to adopt a particular position and a sensor or other device is used to confirm that the patient has complied with that instruction.

In one embodiment of the invention, a system for performing the methods described herein is provided. In one embodiment, a system for treating cardiovascular disease in a medical patient includes an implantable device comprising an electrode, a housing and an impedance measurement module. The electrode is configured to be positioned within the thoracic cavity and configured to deliver at least one electrical pulse. The housing is configured to be positioned in the patient. The impedance measurement module is configured to measure the impedance between the electrode and the housing, which is at least partially metal in some embodiments. The system also includes a signal processing module configured to generate a signal indicative of an appropriate therapeutic treatment to the patient based on the impedance measured by the impedance measurement module. The system further includes a patient advisory module configured to receive the signal generated by the signal processing module and to communicate the signal to the patient. In some embodiments, the implantable device further comprises one or more sensors configured to determine the patient's spatial orientation.

In several embodiments of the systems and methods described herein, the signal indicative of an appropriate therapeutic treatment to the patient is based at least in part on the thoracic impedance and the patient's spatial orientation. In one embodiment, the signal comprises an instruction to the patient. Instructions include, but are not limited to, auditory and visual advice, prescriptions, and/or recommendations. In some embodiments, a mechanical vibration or alarm is used. In one embodiment, the signal comprises at least two distinct instructions to the patient. In other embodiments, the signal comprises more than five, ten or fifteen instructions to the patient. In several embodiments, the instruction comprises advice to the patient that no further action is needed and that the patient is healthy or that the patient's status is unchanged. Alternatively, the instruction comprises advice to the patient to take medication or see a physician. Communication to the patient can also include communication to the patient's clinician.

In some embodiments of the systems and methods described herein, thoracic impedance is measured between a housing and an electrode, wherein the housing is located in the patient and wherein the electrode is located in the thoracic cavity of the patient. The housing comprises metal in some embodiments. In one embodiment, the electrode is positioned within the patient's thoracic cavity and the housing within the patient (for example, in the shoulder region). In one embodiment, at least one electrical pulse is delivered from the electrode and the voltage between the electrode and the housing is measured in response to the electrical pulse. Thoracic impedance is determined, in some embodiments, based on the electrical pulse and the voltage. Voltage can be measured using techniques known in the art, for example, by incorporating circuitry for measuring voltage equivalent or similar to that found in a voltmeter.

In several embodiments, the system and method to detect and treat cardiovascular disease comprise at least one pressure sensor configured to measure left atrial pressure.

### BRIEF DESCRIPTION OF THE DRAWINGS

The structure and operation of the invention will be better understood with the following detailed description of embodiments of the invention, along with the accompanying illustrations, in which:

FIG. 1 illustrates extended dilation of the pulmonary veins, which drain the lung and feed into the left atrium, when left atrial pressure is high.

FIG. 2 illustrates normal dilation of the pulmonary veins when left atrial pressure is within the normal range after a patient's acute episode of heart failure has been substantially treated with diuretics or other medications resulting in a reduction in left atrial pressure.

FIG. 3 illustrates one embodiment of a system for treating cardiovascular disease comprising an implantable module, such as a pacemaker, and an external patient advisory module.

FIG. 4 depicts apparatus suitable for practicing at least one embodiment of the invention.

FIG. 5 depicts an implantable apparatus suitable for practicing another embodiment of the invention.

FIG. 6 is a schematic of one embodiment of the electronics located within the implantable housing of the implantable apparatus illustrated in FIG. 5.

FIG. 7 is a system for treating cardiovascular disease.

FIG. 8 is a block diagram of an external patient advisor/telemetry module for use in one embodiment of the present invention.

FIG. 9 shows a flexible lead. The sheath has been withdrawn to deploy the proximal distal anchors on the right and left atrial sides of the atrial septum, and a physiological sensor is in fluid contact with the patient's left atrium.

FIG. 10 shows a combination of one embodiment of the present invention with an implantable cardiac pacemaker, in which the sensor is implanted in the intra-atrial septum, and the sensor lead also serves as the atrial lead of the pacemaker. A separate ventricular pacing lead is also provided.

FIG. 11 is a schematic diagram depicting digital circuitry suitable for use in one embodiment of the invention.

FIG. 12 is a pacemaker with a digital electrode comprising sense amplifier, pacing/sensing electrode, and defibrillation protection in accordance with one embodiment of the present invention.

FIG. 13 is a pacemaker with a digital electrode as in FIG. 15, wherein the electrode module additionally comprises the charge pump and pacing pulse capacitor in accordance with one embodiment of the present invention.

FIG. 14 is a pacemaker in accordance with one embodiment of the present invention, in which the defibrillation protection is provided within the digital electrode housing.

FIG. 15 is a pacemaker using an analog lead.

FIG. 16 is a pulse timing diagram showing one embodiment for sensing one or more physiological parameters and performing cardiac pacing using a two-conductor digital sensor/pacemaker lead.

### DETAILED DESCRIPTION OF THE PREFERRED EMBODIMENTS

Congestive heart failure (CHF) is a condition in which a patient's heart works less efficiently than it should, and a condition in which the heart fails to supply the body sufficiently with the oxygen-rich blood it requires, either during exercise or at rest. To compensate for this condition and to maintain blood flow (cardiac output), the body retains sodium and water such that there is a build-up of fluid hydrostatic pressure in the pulmonary blood vessels that drain the lungs. As this hydrostatic pressure overwhelms oncotic pressure and lymph flow, fluid shifts from the pulmonary veins into the pulmonary interstitial spaces, and eventually into the alveolar air spaces. This complication of CHF is called pulmonary edema, which can cause shortness of breath, hypoxemia, acidosis, respiratory arrest, and death.

Pressure within the left atrium of the heart is the precursor of fluid accumulation in the lungs, which results in signs and symptoms of acute CHF. Mean left atrial pressure in healthy individuals is normally less than or equal to twelve millimeters of mercury (mm Hg). Transudation of fluid into the pulmonary interstitial spaces can be expected to occur when the left atrial pressure is above about twenty-five mm Hg, or at somewhat more than about thirty mm Hg in some patients with chronic CHF. Pulmonary edema has been found to be predicted by left atrial pressures and less well correlated with conditions in any other chamber of the heart. The early detection and treatment of CHF by measuring left atrial pressure with an implanted pressure sensor has been described by Mann et al. in U.S. Publication No. 2006/0149330 A1, which is hereby incorporated by reference in its entirety.

### I. THORACIC IMPEDANCE

Measurements of thoracic impedance have been experimentally shown to be inversely correlated with left atrial pressure. For example, Ferguson et al. reported a statistically significant inverse correlation between externally measured thoracic impedance and the degree of CHF as seen on plain film chest x-ray (R=0.877, p<0.001), as well as a weaker correlation with pulmonary artery diastolic pressures measured from Swan-Ganz catheters (R=−0.524, p<0.05). Ferguson K L et al., “Correlation of Thoracic Impedance with Pulmonary Artery Pressures in Hypervolemic CHF” Acad. Emerg. Med. 2000 7: 467-a. It should be noted that because impedance and conductance are related by a simple mathematical relationship, a measurement of either impedance or conductance is, in effect, a measure of the other.

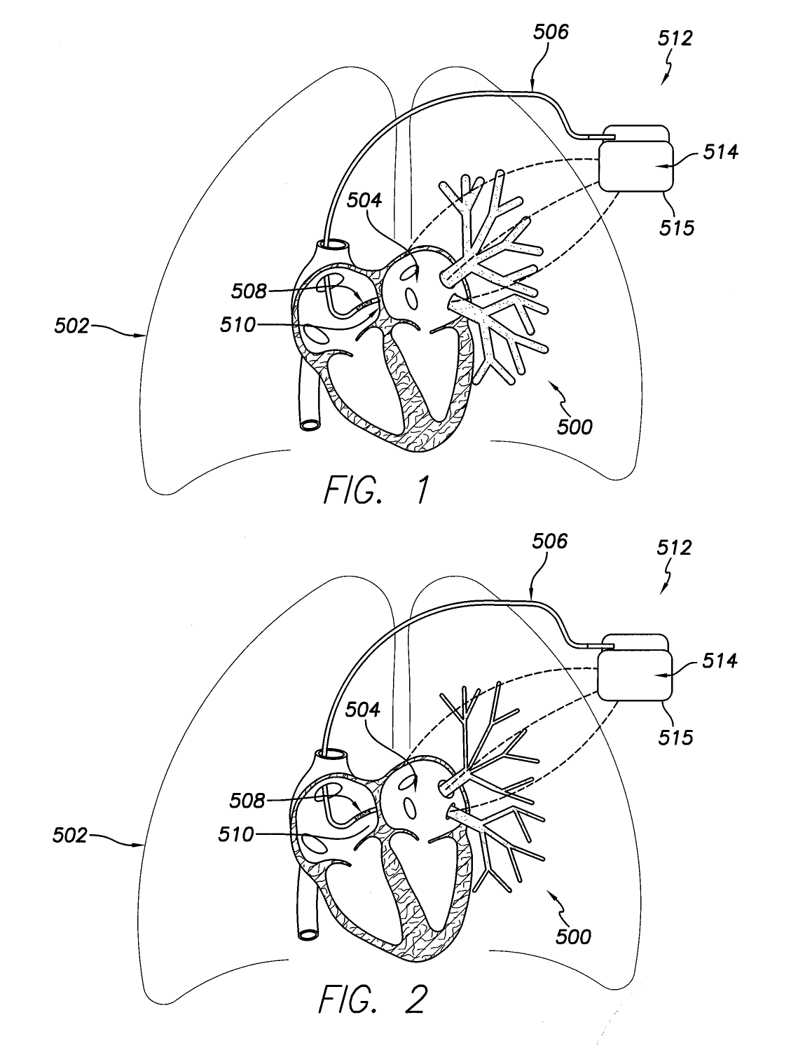

FIG. 1 shows the extended dilation of the pulmonary veins 500, which drain the lung 502 and feed into the left atrium 504, when left atrial pressure is high. In comparison, FIG. 2 shows normal dilation of the pulmonary veins 500 when left atrial pressure is within the normal range after the patient's acute episode of heart failure has been substantially treated with diuretics or other medications resulting in a reduction in left atrial pressure. Because the pulmonary veins 500 are highly compliant structures, even relatively small changes in distending pressure results in large changes in the contained blood volume of the pulmonary veins 500 as shown in FIGS. 1 and 2.

Compared to other tissues in the body, blood is a relatively good conductor of electricity because it is rich in electrolytes. Similarly, fluid in the pulmonary interstitial spaces is also a relatively good conductor. Therefore, when pulmonary blood volume and/or fluid in the pulmonary interstitial space increase, thoracic impedance decreases, and when pulmonary blood volume and/or fluid in the pulmonary interstitial space decrease, thoracic impedance increases. Alternatively, the inverse of impedance is conductance. Restating the relationship in terms of conductance yields the following: when pulmonary blood volume and/or fluid in the pulmonary interstitial space increase, thoracic conductance increases, and when pulmonary blood volume and/or fluid in the pulmonary interstitial space decrease, thoracic conductance decreases. Therefore, measuring the thoracic impedance or conductance provides information on pulmonary blood volume and/or fluid in the pulmonary interstitial space which correlates to left atrial pressure and symptoms of congestive heart failure.

A device that measures thoracic impedance as described herein can function similar to a pressure transducer. Such a device can use changes in electrical impedance or conductance, resulting from changes in blood volume in the pulmonary veins 500 and/or fluid in the pulmonary interstitial space, as a means for detecting left atrial pressure. One benefit of such a device compared with traditional implanted pressure transducers is that there is no need to obtain an atmospheric reference pressure for calibration. This is because a measurement of thoracic impedance is a measure of the distension in the pulmonary veins 500. A measure of the distension in the pulmonary veins 500 is a measurement of the distending pressure of the pulmonary veins 500 which represents the transmural pressure gradient of the pulmonary veins 500. The transmural pressure gradient of the pulmonary veins 500 is the difference between the absolute internal pressure minus the airway pressure, which already takes account of atmospheric pressure.

### A. Impedance Data Acquisition

In one embodiment of the invention, shown in FIGS. 1 and 2, the impedance measuring device 512 comprises a sensor lead 506 with a distal delivery electrode 508 for delivering an electrical pulse or signal. The lead 506 is connected to a housing 514 containing circuitry to measure the electrical impedance or conductance. In the particular embodiment of FIGS. 1 and 2, impedance is measured between the signal delivery electrode 508 and the metallic housing 514, which is acting as an acquisition electrode 515 for the electrical signal. In other embodiments, the acquisition electrode 515 may be separate from the housing 514, either on same lead 506 as the signal delivery electrode or a different lead. In other embodiments, multiple groups of electrodes may be used to make multiple different impedance measurements throughout the thoracic cavity. In some embodiments, to measure thoracic impedance or conductance, the delivery electrode 508 and the acquisition electrode 515 are positioned about the lung 502 such that the electrical circuit between the delivery electrode 508 and the acquisition electrode 515 includes a substantial proportion of lung tissue. The delivery electrode 508 can be positioned, for example, in the atrial septum 510, the coronary sinus, the right ventricle, the left atrium 504 or the lateral epicardial surface of the left ventricle. Using a delivery electrode 508 placed on the epicardial surface facing the lung 502 may be desirable in some embodiments for reducing the contribution of the cardiac blood volume to the impedance or conductance measurement. The acquisition electrode 515 of the impedance measurement device 512 can be positioned, for example, below the subcutaneous fat of the chest wall and above the muscles and bones of the chest. In some embodiments, an acquisition electrode 515 can be positioned outside the heart such that the lung 502 is between the acquisition electrode 515 and the receiving electrode 508. One advantage of this system is that left atrial pressure can be measured without implanting a device directly in the left atrium 504. One of skill in the art will also understand that the locations of the delivery and acquisition electrodes may be reversed, for example, where the housing 514 functions as the delivery electrode 508 by delivering the electrical pulses or signals while the acquisition electrode 515 is provided on a lead 506.

Impedance data acquisition can be accomplished, for example, by delivering pulses of approximately 200 μA and 20 μs pulse width at a frequency of 128 Hz from a delivery electrode 508 and measuring the resulting voltage between the delivery electrode 508 and acquisition electrode 515. The impedance equals the measured voltage divided by the current delivered. Voltage can be measured using techniques known in the art. For example, the impedance measurement device 512 can incorporate circuitry for measuring voltage equivalent or similar to that found in a voltmeter. These pulses generally will not depolarize myocardium, and involve only limited battery drain and have a frequency with an acceptable signal to noise ratio. In some embodiments, the frequency is less than 128 HZ, less than 60 Hz, less than 30 Hz or less than 15 Hz. In some embodiments, the pulse width is less than 20 μs, less than 10 μs, greater than 20 μs, greater than 100 μs, greater than 500 μs or greater than 1000 μs. In some embodiments, the current is less than 1 mA, less than 200 μA, less than 150 μA, less than 100 μA, greater than 200 μA or greater than 300 μA. Alternatively, the delivery electrode 508 can deliver a pulse used to pace or stimulate the heart when warranted by the physiologic conditions of the patient.

Impedance data acquisition can be taken, for example, over approximately a minute long period and then averaged. Averaging can help reduce the effects of the periodic or cyclical physiological phenomena, such as the cardiac cycle and respiratory cycle and the sleep cycle, on impedance measurements. In some embodiments, the measurement period is less than 60 s, less than 30 s, less than 15 s, greater than 60 s, greater than 120 s or greater than 180 s.

Impedance measurements may be taken periodically throughout an extended period of time. In some embodiments, measurements are taken once a day. In other embodiments, measurements are taken multiple times a day, for example hourly. In some embodiments, the hourly measurements are averaged to give a daily average. In other embodiments, measurements are taken less than once a day, for example weekly. Measurements can be taken either when the subject is awake or when the subject is asleep and measurements can be taken at any time throughout the day or night. Measurements can be further averaged, for example, to yield average values while the patient is asleep or awake. The measurement frequency may vary depending upon prior measurements, or based upon patient input of certain symptoms or escalation of symptoms. The acquired impedance measurements may be manipulated to generate a variety of impedance-based measures, including but not limited to averages, standard deviations, and peak and trough measures. These measures may be used to determine individualized threshold levels of certain cardiovascular states. The individualized threshold levels may also be adjusted over time as false-positive or false-negative event states are experienced.

It is also known in the art that electrical impedance changes may be indicative of changes in heart chamber dimensions. An example of a physiological sensor suitable for use in one embodiment of the current invention is described by Alt (U.S. Pat. No. 5,003,976), incorporated by reference herein in its entirety. Alt describes how analyzing the impedance between two intracardiac electrodes may be used to determine changes in cardiac chamber volumes, which under certain circumstances as described above are indicative of changes in chamber pressures, and thus may be used to detect worsening heart failure and guide therapy according to the present invention.

### B. Patient Advisory Module

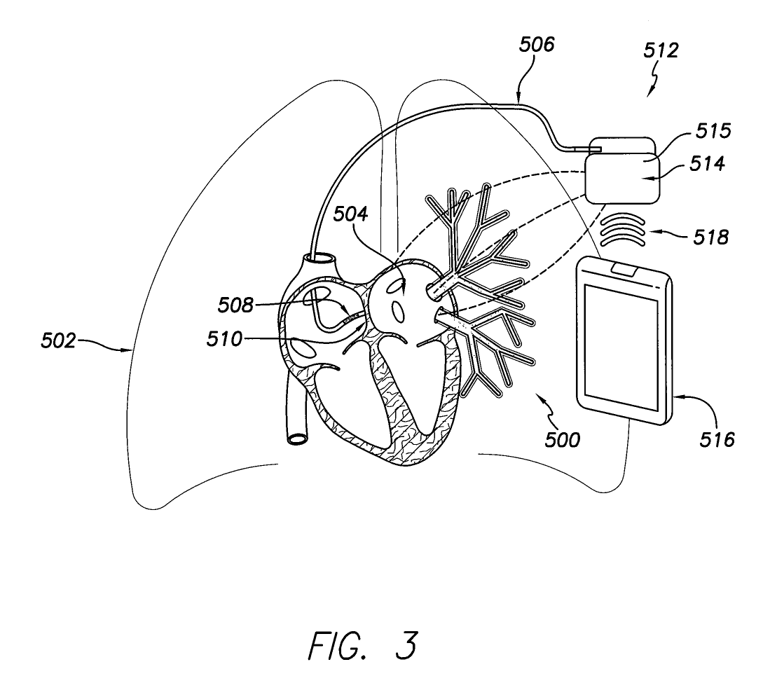

FIG. 3 shows one embodiment of a system for treating cardiovascular disease in general and congestive heart failure in particular. The system comprises a first component comprising an implantable module, such as an impedance measurement device 512 as described above, and a second component comprising an external patient advisory module 516, similar to the embodiment of a patient advisory module described in greater detail below. The patient advisory module 516 is in communication with the impedance measurement device 512 via, for example, radio frequency signals that allow for wireless communication 518 between the modules. The patient advisory module 516 can be a stand alone module or it can be integrated into another device; such as a personal digital assistant, a cell phone, a computer or other consumer electronic device. In some embodiments, the patient advisory module 516 includes a display for displaying data to the patient or physician.

In some embodiments, the patient advisory module 516 is integrated into the impedance data acquisition process. For example, the frequency and timing of the impedance measurements can be programmed and/or stored in the patient advisory module 516. Alternatively, the patient advisory module 516 can be used to program impedance acquisition parameters directly into the impedance measurement device 512.

In addition, in some embodiments the patient advisory module 516 can instruct the patient when to take an impedance measurement and instruct the patient to adopt the correct posture before taking the measurement. Instructing the patient to adopt the correct posture during impedance data acquisition is important because thoracic impedance is affected by posture. Posture affects the distribution of pulmonary fluid, including blood in the pulmonary veins 500 and pulmonary interstitial fluid, which may affect the impedance measurement. For example, when a patient is standing, the lower lobes of the lung 502 have preferential pulmonary venous distention due to a pressure gradient resulting from gravitational effects. Similarly, the lower lobes will also have a preferential accumulation of fluid in the interstitial spaces. When the patient is recumbent, the gravity induced pressure gradient is such that the portions of the lung 502 toward the back or posterior of the thorax preferentially accumulate pulmonary venous blood and interstitial fluid. Depending on the placement of the delivery electrode 508 and the acquisition electrode 515, the impedance measurement may vary with the patient's posture due to posture relate effects on pulmonary fluid distribution.

In order to reduce posture-related artifacts in the impedance measurements, the patient advisory module 516 can instruct the patient to adopt a particular posture before impedance data acquisition. For example, the patient advisory module 516 can instruct the patient to lie supine for 5 minutes before taking a 20 second reading of impedance. The particular instructions to the patient may be programmable, to accommodate physiological and physical differences between individual patients. In some embodiments, the patient can input his posture into the patient advisory module 516 before, after or during impedance data acquisition. This feature is useful if the patient is unable to comply with the posture request or inadvertently adopted the incorrect posture.

Alternatively, in some embodiments either the impedance measurement device 512 or a third component can comprise a sensor that detects the patient's posture. Examples of such a sensor include an accelerometer, a multiaxis tiltometer or a pressure sensor that measures a gravity-induced pressure gradient. Once the patient's posture is determined, the patient's posture can be communicated to the patient advisory module 516 which can, for example, notate the patient's posture at the time of each impedance measurement.

Respiration can also have an effect on impedance measurements because the volume of air, a relatively good insulator, in the lungs 502 changes during the respiratory cycle. At the end of inhalation, the volume of air in the lungs 502 is relatively high, which can increase thoracic impedance. Conversely, at the end of exhalation, the volume of air in the lungs 502 is relatively low, which can decrease thoracic impedance.

The patient advisory module 516 can also instruct the patient to take a certain medication or alter the dosage of such medication based on the acquired impedance measurements and a physician prescribed algorithm. For example, if the patient advisory module 516 detects lower impedance measurements indicative of increased left atrial pressure and congestive heart failure, the patient advisory module 516 can instruct the patient to either take or increase the dosage of diuretics and/or vasodilators to decrease the patient's blood pressure. If the medications are able to return impedance measurements to a normal range, indicating normal left atrial pressure, the patient advisory module 516 can scale back the medication regimen, if desired.

Additional instructions provided by the patient advisory module 516 to the patient based on the acquired impedance measurements include instructions on diet, sodium intake, contacting a physician and other activities. For example, high sodium intake may lead to increased water retention and increased blood pressure. By instructing the patient to reduce sodium intake, blood pressure may be lowered, thereby returning impedance measurements to a normal range. In addition, if the patient does not respond to medication, diet or sodium intake controls, the patient advisory module 516 can instruct the patient to see a physician. Furthermore, the patient advisory module 516 can give multiple instructions to the patient simultaneously. For example, the patient advisory module 516 can instruct the patient to increase his medication, reduce sodium intake and see a physician. The patient can input into the patient advisory module 516 which, if any, of the instructions was followed or notate any deviation from the instructions.

In some embodiments, the patient advisory module 516 can provide the patient and/or the physician with patient data, such as the recorded impedance data, the instructions provided to the patient and any notations made by the patient. In some embodiments, the patient and/or the physician can obtain the information from the patient advisory module 516 by viewing a display on the patient advisory module 516. In some embodiments, the patient and/or the physician can obtain the information from the patient advisory module 516 using a wire or cable to download the information from the patient advisory module 516 to, for example, a computer. Alternatively, the patient advisory module 516 can communicate this information wirelessly. In some embodiments, the information can be remotely transmitted to the patient and/or the physician via, for example, the Internet, a phone line, a fiber optic network or a cellular network.

When the physician is in communication with the patient advisory module 516, the physician can also modify the parameters, instruction set and/or algorithms of the patient advisory module 516. For example, the physician can increase the frequency of the impedance measurements or change the algorithms controlling drug dosing in response to a patient's changing physiologic state.

In other embodiments, the impedance measurement device and the patient signaling device are permanently implanted, and the patient is signaled using at least two distinguishable stimuli, such as distinguishable sequences of vibrations, acoustic signals, or mild electrical shocks, perceptible by the patient.

Further details of a thoracic impedance-based system for assessing and treatment congestive heart failure are provided below.

### II. THE THORACIC IMPEDANCE SYSTEMS

In one embodiment, the invention comprises a system and method for detecting and treating cardiovascular disease (such as CHF) in a medical patient based on the patient's thoracic impedance and spatial orientation (such as the patient's location and/or position).

In another embodiment of the invention, a method of detecting and treating cardiovascular disease includes the steps of generating a sensor signal indicative of a thoracic impedance, generating a processor output indicative of a treatment to a signaling device, and providing at least two treatment signals to the medical patient. The processor output is based at least in part on the sensor signal. Each treatment signal is distinguishable from one another by the patient, and is indicative of a therapeutic treatment. At least one signal is based at least in part on the processor output. The patient's spatial orientation is also ascertained in several embodiments, and the treatment signal is based on both the thoracic impedance and the spatial orientation. In several embodiments, atrial pressure is measured in addition to thoracic impedance.

### A. Stand-Alone Thoracic Impedance System

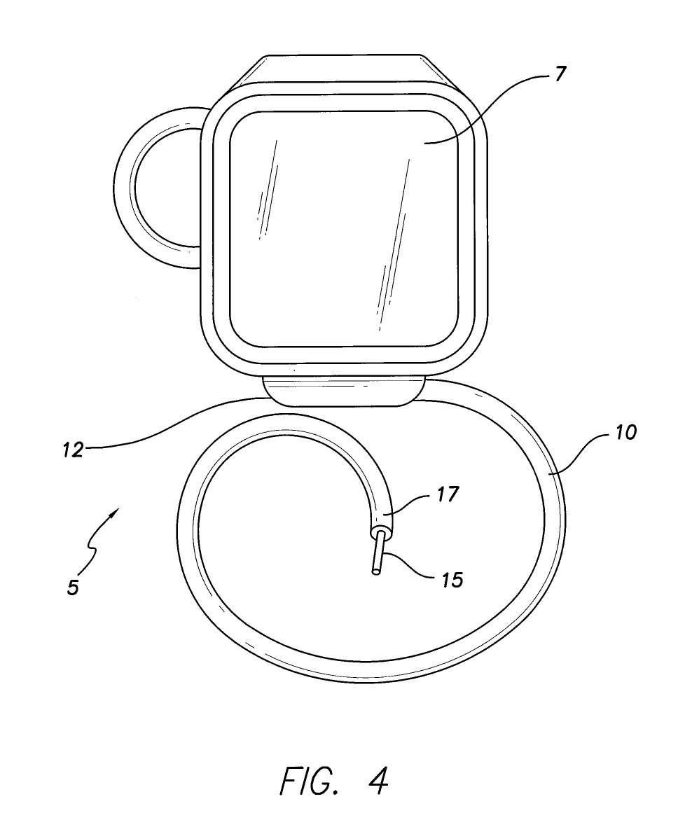

FIG. 4 shows a system for treating cardiovascular disease, such as congestive heart failure, which includes an implantable impedance device 5 in accordance with one embodiment of the invention. The implantable impedance device 5 includes a housing 7 and a flexible, electrically conductive lead 10. The lead 10 is connectable to the housing 7 through a connector 12 that may be located on the exterior of the housing. The flexible lead 10 is also generally similar to leads used in defibrillator and pacemaker systems, except that an impedance electrode 15 for delivering an electrical pulse or current is disposed on the lead 10, typically on the distal end 17 opposite end from the connector 12 on the housing 7. The flexible lead 10 may also contain the impedance acquisition electrode, or other types of sensors to measure one or more other parameters. The housing 7 may include a signal processor (not shown) to process the signal detected by the acquisition electrode. The housing may also include an electrical acquisition module for measuring the electrical signal emitted from the impedance delivery module 15. Where the housing 7 comprises a metallic shell, the sensor module may utilize the shell itself as the acquisition interface, but in other embodiments, a dedicated acquisition interface on the shell may be provided. In addition, the housing 7 may include telemetry or signaling devices (not shown), to either communicate with an external device, or signal the patient, or both. The elements inside the housing 7 may be configured in various ways, as described below, to communicate to the patient a signal, such as a treatment signal, indicative of an appropriate therapy or treatment based at least in part on one or more of the measured physical parameters.

One skilled in the art will appreciate that the lead can be of any length appropriate to connect the impedance signal delivery module. In another embodiment, the lead length is zero, such that the sensor package is configured to occupy substantially the same location.

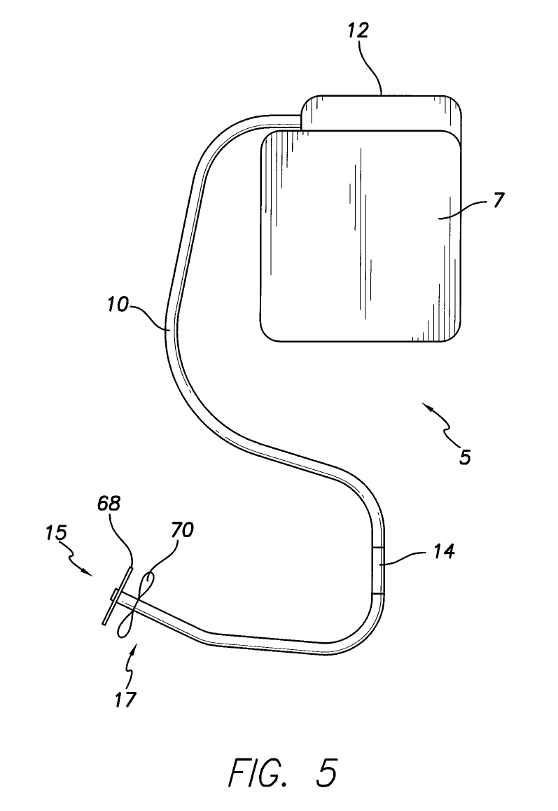

In some embodiments, the lead 10 is positioned at sites typically associated with pacemaker or defibrillator leads, such as the coronary sinus, right ventricle and the superior vena cava, as cardiologists already have in placing leads at such sites. In some embodiments, however, the electrode is configured for implantation at other intracardiac sites or at extracardiac sites. FIG. 5 shows one embodiment in which the impedance signal delivery electrode 15 has distal 68 and proximal 70 anchoring mechanisms configured to anchor the delivery electrode 15 to a organ or a lumen wall. Some embodiments of the distal 68 and proximal 70 anchoring mechanisms that may be used with the invention are described in greater detail in U.S. Pat. No. 7,149,587, herein incorporated by reference in its entirety. An electrode implanted at the atrial septum, for example, may provide an impedance measurement that correlates better with LAP or CHF status due to its proximity to the pulmonary arteries and veins. In another example, the electrode and its anchoring mechanism may be configured for deployment about the pericardium. The electrical pathway between the electrode 15 at an extra-cardiac site and the housing 7 may be more indicated of pulmonary fluid status as it reduces the effect of fluid within the cardiac chambers on the impedance measurement.

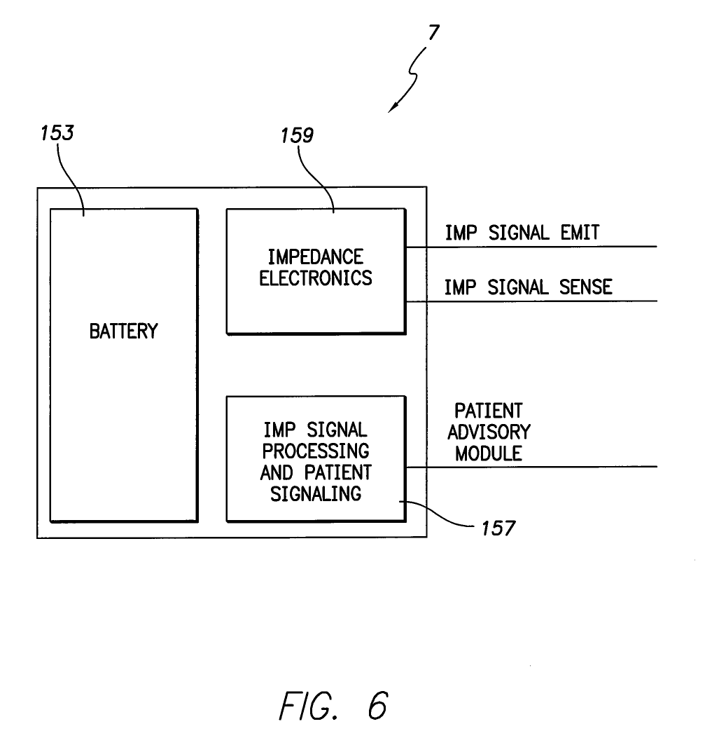

The housing 7 typically contains electronics (not shown) and other components (not shown) for communicating with an external module ((not shown). One embodiment showing the contents of the housing 7 is illustrated in FIG. 6. As shown in FIG. 6, in one embodiment, housing 7 includes a power supply 153, an impedance system 159, and a signal processing and patient signaling modules 157. The impedance system 159 is configured to provide an electrical signal to the patient's thoracic cavity, and sense the electrical signal from implanted sensors or housing. The signal processing module 157 may also be configured to control multiple implanted impedance components, or a sensor package or module, as described in greater detail herein.

As mentioned previously, in some embodiments, the housing 7 is a metallic housing that may be used as the acquisition electrode for sensing the electrical pulse used to measure thoracic impedance. In other embodiments, however, a separate lead electrode remotely positioned from the housing 7 may be used. One of skill in the art will also understand that combinations of multiple electrode and sensors leads may be used to generate a composite signal to measure thoracic impedance. There need not be a one-to-one correspondence between the number of electrical pulse emitting leads and the number of sensing leads, as any one electrical pulse may be sensed by a plurality of sensing modules and vice versa. The individual components comprising a composite thoracic impedance measure may be weighted to improve the correlation between the impedance measure and left atrial pressure or other measure indicative of pulmonary congestion.

As described above and in other embodiments herein, a system for detecting and treating cardiovascular disease in a medical patient may include, in addition to the impedance measurement system, at least one physiological sensor used to generate a signal indicative of an anatomic or physiological parameter on or in the patient's body. The system includes signal processing apparatus operable to generate a signal, such as a processor output, indicative of an appropriate therapeutic treatment, which in one embodiment is based at least in part upon the signal generated by the physiological sensor.

As mentioned previously, in some embodiments, the sensor system comprises a motion or position sensor that may be used to determine assess patient body orientation. Because body fluid, including fluid causing pulmonary congestion, is gravity-dependent, measures indicative of left atrial pressure and pulmonary congestion may be increased when the patient is recumbent. Distinguishing between changes resulting from body position and changes in overall fluid status from heart failure may be used to reduce false-positive results or increase the sensitivity for detecting heart failure. Signals from the position sensor may be monitored continuously or at appropriate intervals. Information is then communicated to the patient corresponding to appropriate physician-prescribed drug therapies. In one embodiment, the information is the treatment signal. In many cases, the patient may administer the drug therapies to him or herself without further diagnostic intervention from a physician.

In another embodiment, the physiological sensor is a pressure transducer that is positioned to measure pressures within the patient's left atrium. Signals from the pressure sensor may be monitored continuously or at appropriate intervals. The thoracic impedance information may be correlated to the pressure information from this sensor or from periodic pressure determinations, from cardiac ultrasound for example, so that the impedance measurements can be translated into atrial pressure information that are more familiar to physicians. In other embodiments, a composite measure for assessing CHF status may be generated using a combination of pressure data and thoracic impedance data.

Non-pressure physiologic parameters may be used in other embodiments. In one embodiment, the internal electrocardiogram (known as the IEGM) is sensed at one or more locations. In a further embodiment, the IEGM is processed to obtain one or more medically useful parameters. These parameters include, but are not limited to, heart rate, the timing of atrial and ventricular depolarization, the time interval between atrial and ventricular depolarization (known in the art as the A-V interval), the duration of ventricular depolarization (known in the art as the Q-T interval), ST segment changes to detect acute ischemia, and spectral analysis to detect t-wave alternans (a known indicator of life threatening arrhythmias), all of which are familiar to those skilled in the art. Embodiments incorporating cardiac rhythm management components, such a pacemaker or a defibrillator, are described in greater detail below.

Casscells III et al. (U.S. Pat. No. 6,454,707), incorporated by reference herein, describe a method and apparatus for predicting mortality in congestive heart failure patients by monitoring body temperature and determining whether a downward trend in temperature fits any predetermined criteria. In another embodiment, core body temperature is measured at the site of a measurement module located anywhere within the heart, heart chambers, great vessels, or other locations within the thorax known in the medical arts to maintain a temperature related in a predictable way to core body temperature.

Regional elevations in temperature are known to those skilled in the art of temperature physiology to occur in the presence of inflammation. Inflammation occurs in the heart in many cardiovascular diseases. A temperature sensor of sufficient precision residing in proximity to the walls of the heart may detect regional elevations in temperature due to local tissue inflammation. Inflammatory cardiac conditions may also be associated with a rise in left atrial pressure. In one embodiment of the present invention, an implanted monitoring system that measures both local tissue temperature with a precision of approximately 0.1° C. and a parameter indicative of left atrial pressure can be used to diagnose active cardiac inflammation and concomitant cardiac dysfunction.

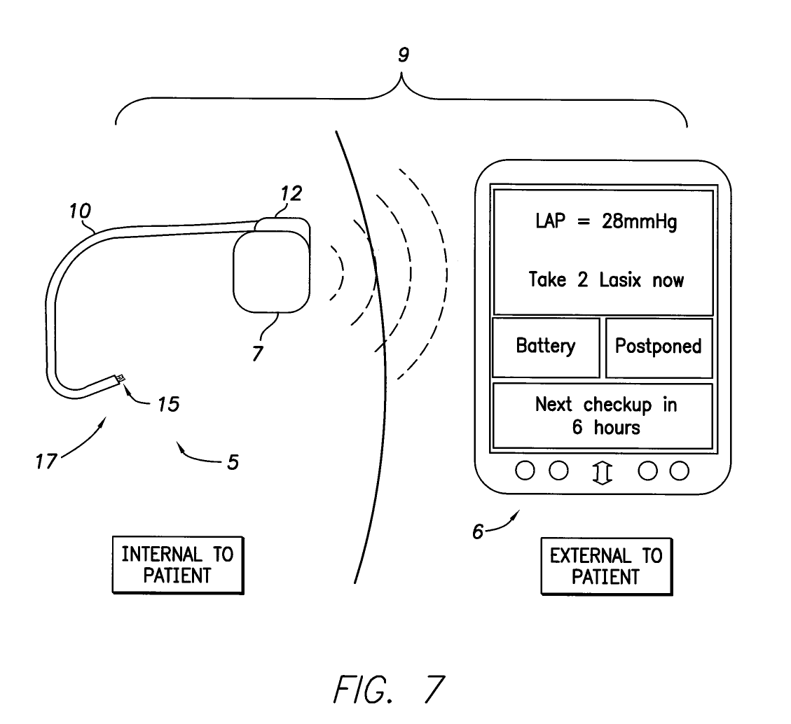

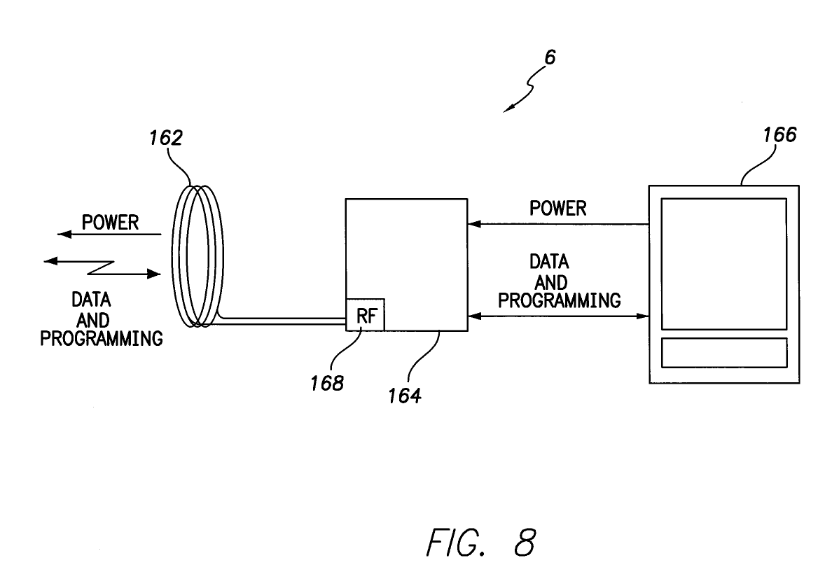

FIG. 7 depicts one embodiment of a system for treating cardiovascular disease 9. The system 9 includes a first component comprising an implantable impedance device 5, such as that described with reference to FIG. 5, and a second component comprising an external patient advisory module 6, such as that described below with reference to FIG. 8. During system 9 operation, electrical pulses or signals are carried by a lead 10 between an impedance signal package 15 located near the distal end 17 of the lead 10, and a housing 7 of an implantable module 5. The circuitry inside the housing 7 includes an antenna coil (not shown). In this embodiment, signals are communicated between the implantable impedance device 5 and an external device, such as a patient advisory module 6, via the antenna coil of the housing 7 and a second external coil (not shown) coupled to the external device 6.

In one embodiment, as shown in FIG. 6, the housing 7 contains a battery 153 that powers the implantable impedance device 5. In another embodiment, the implanted impedance device 5 receives power and programming instructions from the external device 6 via radio frequency transmission between the external and internal coils. The external device 6 receives signals indicative of one or more physiological parameters from the implanted device 5 via the coils as well. One advantage of such externally powered implantable device 5 is that the patient will not require subsequent surgery to replace a battery. In one embodiment of the invention, power is required only when the patient or the patient's caregiver initiates a reading. In other situations, where it is desired to obtain impedance or physiological information continuously, or where it is desired that the implanted impedance device 5 also perform functions with higher or more continuous power requirements, the housing 7 may also contain one or more batteries. As described below, the housing 7 may also contain circuitry to perform additional functions that may be desirable.

FIG. 8 schematically shows one embodiment of the second component of the system, a patient advisory module 6. In one embodiment, the patient advisory module 6 includes a Palm-type computing device with added hardware and software. Referring to FIG. 8, a patient advisory module 6 includes a radio frequency telemetry module 164 with an associated coil antenna 162, which is coupled to a processing unit 166. In one embodiment, the processing unit 166 includes a Palm-type computer, personal digital assistant (PDA), or cell phone, as is well known to those of skill in the art. In one embodiment, the patient advisory module 6 powers the implanted apparatus (not shown) with the telemetry hardware module 164 and coil antenna 162. In another embodiment, the patient advisory module 6 receives impedance or other physiological signals from the implanted first component of the system by wireless telemetry through the patient's skin.

The signal processing unit can be used to analyze physiologic signals and to determine physiologic parameters. The patient advisory module 166 may also include data storage, and a sub-module that contains the physician's instructions to the patient for therapy and how to alter therapy based on changes in physiologic parameters. The parameter-based physician's instructions are referred to as “the dynamic prescription,” or DynamicRx™. The instructions are communicated to the patient via the signaling module 166, or another module. The patient advisory module 166 is located externally and used by the patient or his direct caregiver. It may be part of system integrated with a personal digital assistant, a cell phone, or a personal computer, or as a “Stand-Alone” device (e.g., in one embodiment, the HeartPOD™ diagnostic and therapeutic drug management system) without combination with CRM apparatus, described in greater detail below. In one embodiment the patient advisory module communicates with a remote site such as a doctor's office, clinic, hospital, pharmacy, or database. Revised patient instructions including the parameter-based dynamic prescription can be communicated back to the patient advisory module. This can be performed remotely via hard-wired telephone or fiberoptic cable networks or wirelessly using a host of communication technologies currently available. Data may be communicated in either direction and the Internet may be in part the conduit for such communication.

In one embodiment, the impedance and other physiologic signals are analyzed and used to determine adjustable prescriptive treatment instructions that have been placed in the patient advisory module 6 by the patient's personal physician. Communication of the prescriptive treatment instructions to the patient may appear as written or graphic instructions on a display of the patient advisory module 6. These treatment instructions may include what medications to take, dosage of each medication, and reminders to take the medications at the appropriate times. In one embodiment, the patient advisory module 6 displays other physician-specified instructions, such as “Call M.D.” or “Call 911” if monitored values become critical.

For example, some embodiments of the invention comprise an automatic therapy regime based upon a programmed dynamic prescription. “Dynamic prescription,” as used herein, shall mean the information that is provided to the patient for therapy, including instructions on how to alter therapy based on changes in the patient's physiologic parameters. The instructions may be provided by a physician, practitioner, pharmacist, caregiver, automated server, database, etc. The information communicated to the patient includes authorizing new prescriptions for the patient and modifying the patient's medicinal dosage and schedule. The “dynamic prescription” information also includes communicating information which is not “prescribed” in its traditional sense, such as instructions to the patient to take bed rest, modify fluid intake, modify physical activity, modify nutrient intake, modify alcohol intake, perform a “pill count,” measure additional physiological parameters, make a doctor's appointment, rush to the emergency room, call the paramedics, etc. One skilled in the art will understand that numerous other instructions may be beneficially provided to the patient predicated at least in part upon measurement of one or more physiological parameters in accordance with various embodiments of the present invention.

In one embodiment, the treatment signal may be the numerical representation of a parameter indicative of fluid pressure in the left atrium, such as thoracic impedance. As mentioned previously, in some embodiments, correlative functions may be used to represent a numerical value, such as thoracic impedance, in terms that are more familiar to physicians, such as left atrial pressure or pulmonary artery wedge pressure. Physician specified treatments would be supplied to the patient in the form of a decoding reference providing different treatment instructions for specified ranges of left atrial pressure and/or thoracic impedance. Such a decoding reference could be written or printed instructions on a card that the patient keeps for reference, or directly reported to the patient through the visual display of the system. For example, a mean left atrial pressure (LAP) of 15 mm Hg would indicate the same treatment as a mean LAP of 16 mm Hg, both values being in a range indicating that the patient's heart failure is well compensated. An LAP of 25 mm Hg however would indicated decompensated CHF and would decode as different therapeutic instructions aimed at recompensating the state of CHF.

A third component of this system embodiment is designed for physician use. The third component is used to program the dynamic prescription and communicate it or load it into the patient advisory module 166. The third module may also contain stored data about the patient, including historical records of the impedance signals and derived parameters transmitted from the patient implant and signaling modules. The third component may also communicate with external databases. In one embodiment, the third component is a physician input device, and includes a personal computer, a PDA, a telephone, or any other such device as is well known to those of skill in the art also comprising specific third component software or firmware programs.

1. Implant Therapy Units

In one embodiment of the present invention, the first implant module (for example, implantable impedance device 5 of FIGS. 4 and 5) may also contain an implant therapy unit, or ITU. The ITU generates an automatic therapy regime based upon the programmed dynamic prescription. The therapy may include, but is not limited to, a system for releasing bioactive substances from one or more implanted reservoirs, controllers for ventricular or other types of cardiac assist devices, and a pacemaker or defibrillator system.

Some embodiments of the invention comprise an implant therapy unit, including but not limited to a system for releasing bioactive substances from implanted reservoir(s), a system for controlling electrical pacing of the heart, and cardiac assist devices including pumps, oxygenators, artificial hearts, cardiac restraining devices, ultrafiltration devices, intravascular and external counterpulsation devices, continuous positive airway pressure devices, and a host of related devices for treating cardiovascular conditions where knowledge of the thoracic impedance and/or left atrial pressure would be beneficial for optimal therapy delivery.

In one embodiment, dosimetry for multiple drugs or other associated therapeutic devices is relayed based on parameter values as input to a parameter-driven prescription. In one embodiment, the system essentially replicates, in the home setting, the way inpatients are managed based on their doctor's standing orders in the Intensive Care Unit (ICU) of a hospital. In the ICU, nurses periodically look at real-time physiologic values from diagnostic catheters, and administer medications based on predetermined orders by the patient's attending physician. One embodiment of the present invention accomplishes the same thing. In one embodiment, wireless communications technology is integrated with diagnostic and treatment methods that are well established in cardiology. As such, the system is designed to be convenient and time-efficient for both the patient and his physician. The combination of monitoring key physiologic parameters and the patient's own physician's prescription drive a real-time feedback loop control system for maintaining homeostasis. Thus, in one embodiment, the system comprises an integrated patient management system tightly and directly linking implantable sensor diagnostics with pharmacologic and other therapies. As a result, this therapeutic approach enables better, more cost effective care, improves out-of-hospital time, and empowers patients to play a larger and more effective role in their own healthcare.

In one embodiment, the impedance information is used to adjust pacing therapy such that pacing is performed only when needed to prevent worsening heart failure. One skilled in the art will appreciate that many systems or devices that control the function of the cardiovascular system may be used in accordance with several embodiments of the current invention. Combined impedance measurement systems and cardiac rhythm management devices are discussed in greater detail below.

In one embodiment, a portable system for continuously or routinely monitoring one or more parameters indicative of the condition of a patient is provided. Depending upon changes in the indicated condition, the system determines, based on parameter-driven instructions from the patient's physician, a particular course of therapy. The course of therapy is designed to manage or correct, as much as possible, the patient's chronic condition. In one embodiment, the system communicates the course of therapy directly to the patient or to someone who assists the patient in the patient's daily care, such as, for example, but not limited to, a spouse, an aid, a visiting nurse, etc. In one embodiment of the invention, the advisory module 6 is programmed to signal the patient when it is time to perform the next cardiac status measurement and to take the next dose of medication. It will be recognized by those skilled in managing CHF patients that these signals may help the many patients who have difficulty taking their medication on schedule. Although treatment prescriptions may be complex, one embodiment of the current invention simplifies them from the patient's perspective by providing clear instructions. To assure that information regarding the best treatment is available to physicians; professional cardiology organizations such as the American Heart Association and the American College of Cardiology periodically publish updated guidelines for CHF therapy. These recommendations can serve as templates for the treating physician to modify to suit individual patient requirements. In one embodiment, the device routinely uploads data to the physician or clinic, so that the efficacy of the prescription and the response to parameter driven changes in dose can be monitored. This enables the physician to optimize the patient's medication dosage and other important treatments without the physician's moment-to-moment intervention.

The embodiments summarized above and described in greater detail below are useful for the treatment of cardiovascular disease, including congestive heart failure (CHF). CHF is an important example of a medical ailment currently not treated with timely, parameter-driven adjustments of therapy, but one that the inventors believe could potentially benefit greatly from such a strategy. Patients with chronic CHF are typically placed on fixed doses of an average of six drugs to manage the disease. The drug regimen commonly includes but is not limited to diuretics, vasodilators such as ACE inhibitors or A2 receptor inhibitors, beta-blockers such as Carvedilol, neurohormonal agents such as spironolactone, and inotropic agents usually in the form of cardiac glycosides such as, for example, digoxin. In addition, patients typically are taking other cardiovascular drugs to limit disease progression, symptoms or complications. Examples include ‘statins’ to lower cholesterol, nitrate to relieve chest pain, and aspirin or warfarin to prevent clotting.

2. Implantation and Anchoring

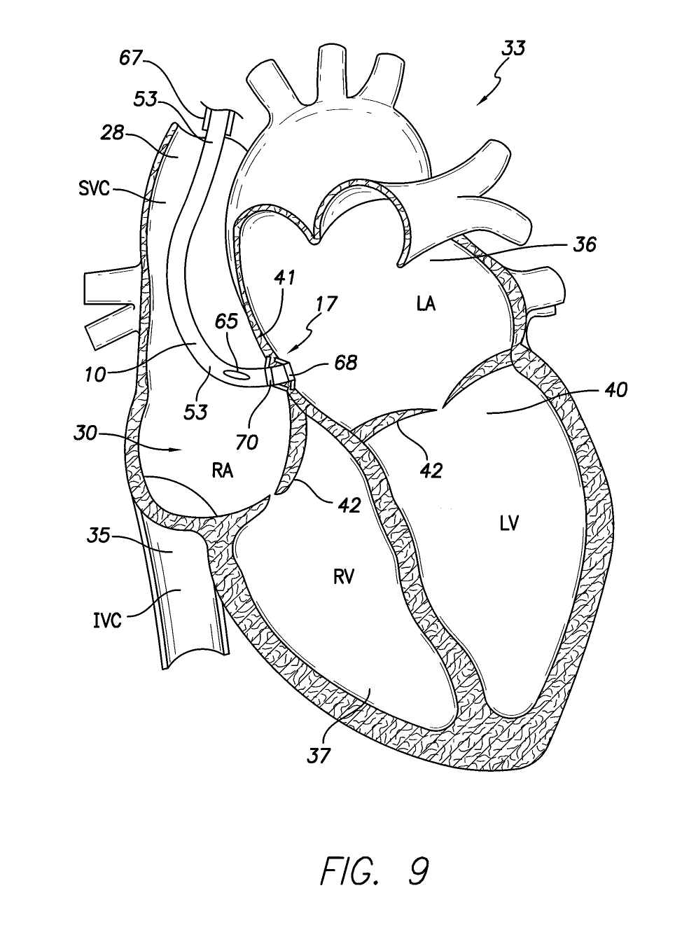

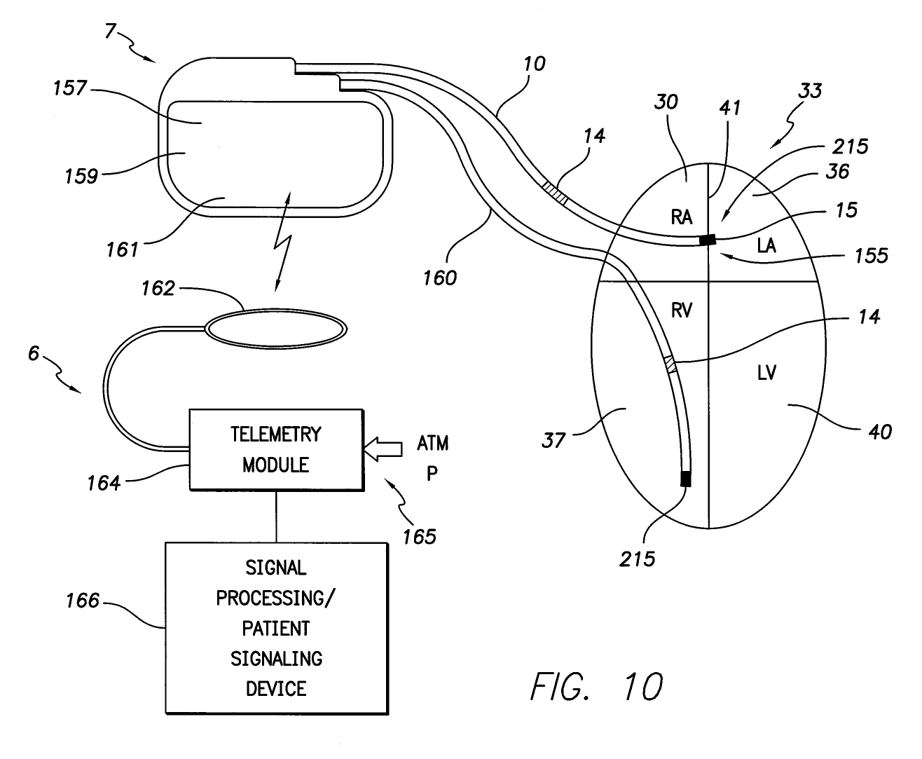

As mentioned previously, in some embodiments of the invention, the electrode leads of the implantable impedance device are implanted in traditional pacemaker/defibrillator locations, such as the coronary sinus, the right ventricle and the superior vena cava. In other embodiments, the implantable impedance device is surgically or minimally invasively implanted in the patient at other sites, such as the pericardium or the intra-atrial septum. The implanted lead may contain an impedance electrode or a physiologic sensor of the implanted device. As illustrated in FIG. 9, one example of implanting a lead 10 into the intra-atrial septum 41 comprises approaching the left atrium 36 through the right atrium 30, penetrating the patient's atrial septum 41 and positioning the distal end 17 of the lead 10 in the atrial septum 41, on the septal wall of the left atrium 36, or inside the patient's left atrium 36. It will also be apparent that, in several embodiments, a similar sensor/lead system can be inserted through an open thoracotomy or a minimally invasive thoracotomy, with the anchoring system fixating the sensor/lead to a location such as the free wall of the left atrium, the left atrial appendage, or a pulmonary vein, all of which provide access to pressures indicative of left atrial pressure.

One skilled in the art will understand that alternative lead routes and exit sites from the venous system may also be used. One class of alternative implantation methods consists of surgical implantation through the wall of the heart, either directly into the left atrium through the left atrial free wall or left atrial appendage, into the left atrium via a pulmonary vein, into the left atrium through the intra-atrial septum via the right atrial free wall, or directly into a pulmonary vein.

3. Coatings, Polishing, and Drug Eluting Surfaces

In one embodiment, a coating inhibits or minimizes the formation of undesirable fibrous tissue, while not preventing the beneficial growth of an endothelial covering. Coatings with these properties are well known in the art of implanting medical devices, particularly intravascular stents, into the blood stream. Surface coating materials include, but are not limited to, paralene, PVP, phosphoryl choline, hydrogels, albumen affinity, and PEO.

In one embodiment, at least some areas of the impedance signal package are electropolished. Electropolished surfaces are known by those skilled in the art to reduce the formation of thrombosis prior to endothelialization, which leads to a reduced burden of fibrotic tissue upon healing. Metallic intracoronary stents currently approved for clinical use are electropolished for this purpose.

Release of antiproliferative substances including radiation and certain drugs are also known to be effective in stenting. Such drugs include, but are not limited to, Sirolimus and related compounds, Taxol and other paclitaxel derivatives, steroids, other anti-inflammatory agents such as CDA, antisense RNA, ribozymes, and other cell cycle inhibitors, endothelial promoting agents including estradiol, antiplatelet agents such as platelet glycoprotein IIb/IIIa inhibitors (ReoPro), anti-thrombin compounds such as heparin, hirudin, hirulog etc, thrombolytics such as tissue plasminogen activator (tPA). These drugs may be released from polymeric surface coating or from chemical linkages to the external metal surface of the device. Alternatively, a plurality of small indentations or holes can be made in the surfaces of the device or its retention anchors that serve as depots for controlled release of the above mentioned antiproliferative substances, as described by Shanley et al. in U.S. Publication No. 2003/0068355, published Apr. 10, 2003, incorporated by reference herein.

4. Signal Processing Apparatus

In one embodiment, the signal processing apparatus of the present invention receives signals from the one or more sensors, and processes them together with stored parameters relevant to the patient's medical management. In one embodiment, the result of this processing is a signal indicative of the appropriate therapeutic treatment or course of action the patient or an immediate personal care giver can take to manage or correct, as much as possible, the patient's condition. In one embodiment, the signal processing apparatus is located outside the patient's body. In one embodiment, signals from one or more permanently implanted physiological sensors are received by the external signal processing apparatus by wireless telemetry. In one embodiment, certain signal processing is performed within the one or more individual sensor devices prior to the signal being sent to the signal processing apparatus. In one embodiment, for each patient-specific programmed treatment range the patient's physician stores in the signal processing apparatus an indication of the appropriate therapeutic treatment or action the patient should take to manage or correct, as much as possible, the patient's condition. A signal indicative of the physician-prescribed therapeutic action corresponding to the patient-specific range into which the measured physiologic parameter falls is then sent to a patient signaling device.

In another embodiment of the invention, the signal processing apparatus is essentially permanently implanted within the body, in either the same or a different location as the one or more physiological sensors. In another embodiment, the impedance and physiologic sensors may be in wireless communication with the signal processing apparatus. The lead can be coupled to an antenna for wireless transmission or to additional implanted signal processing or storage apparatus.

5. Interpretation of Signals

In one embodiment of the present invention, patients are diagnosed based upon the interpretation of signals generated by one or more impedance measurement systems. One skilled in the art will understand that other interpretations may be used in accordance with various embodiments of the current invention. Further, one skilled in the art will understand that normal ranges of the various physiologic parameters measured in several embodiments of the current invention can be found in cardiology textbooks or reference books. Additionally, it may be useful to compare patient parameters within the same patient by ascertaining initial baseline values and comparing these baseline numbers to values generated at some later desired time. This may be particularly useful in determining progression of disease and response to treatment.

In several embodiments, sensors in addition to the thoracic impedance sensor are used. Additional sensors provide further refined diagnostic modes capable of distinguishing between different potential causes of worsening cardiovascular illness, and then of signaling an appropriate therapeutic treatment depending upon the particular cause for any particular occurrence.

In another example of the usefulness of additional physiological signals is to distinguish between pulmonary congestion caused by worsening CHF and that caused by a respiratory infection. In a further embodiment, core body temperature is used together with thoracic impedance to allow the early detection of fever associated with infection. It is well known that core body temperature often becomes elevated hours to days prior to symptomatic fever associated with infection-related pulmonary congestion. In one embodiment, increased core temperature in the presence of stable thoracic impedance would trigger a message to the patient not to increase the dosage of oral diuretic despite symptoms of increasing congestion, and to consult with the physician.

B. Combination with Other Devices

In further embodiments of the current invention, the system and method for detecting and treating cardiovascular disease includes a cardiac rhythm management (CRM) module. In some embodiments, though, the cardiac rhythm management module includes related devices that do not electrically depolarize all or some portion of the heart muscle to manage a cardiac rhythm or the synchrony of depolarization, but are used to perform some other therapeutic function. For example, delivering electrical stimuli to cardiac muscle during the refractory period after depolarization may increase the strength of cardiac contraction, a phenomenon known as an ‘ionotropic’ effect. This may be helpful in generating more cardiac output in CHF patients with low cardiac output.

It will be clear to those skilled in the art that many patients who would benefit from several embodiments of the present invention would also benefit from an implantable CRM apparatus such as a cardiac pacemaker. In one embodiment, the present invention is combined with an implantable CRM apparatus generator. In one embodiment, the flexible lead on which an impedance electrode or a physiological sensor is disposed also serves as the sensing or pacing lead of an implantable rhythm management apparatus. In this case, conductors within the lead provide for EKG sensing, powering of the physiological sensor, data communication for the physiological sensor, and pacing stimulus.

Although the impedance measurement component of the implantable device may operate independently of the CRM component, in other embodiments, the impedance measurement device is functionally integrated with another implantable a pacemaker or defibrillator component. Thus, the information produced by the impedance measurement component may used by the integrated device to control atherapeutic function provided by the non-impedance component, as described below.

1. Combination with Cardiac Rhythm Management (CRM) Apparatus

Many patients who might benefit from impedance measurement device described above would also be likely to benefit from an implantable CRM apparatus for therapy of brady- or tachy-arrhythmia in the setting of CHF. Examples of such CRM devices include single or multichamber cardiac pacemakers; automatic implanted cardiac defibrillators; combined pacemaker/defibrillators; biventricular pacemakers; and three-chamber pacemakers, all well known to those skilled in the art. In these patients, it would be beneficial to combine several embodiments of the implantable impedance device with such a CRM device. This combination would have the advantage that certain components of both systems could be shared, reducing cost, simplifying implantation, minimizing the number of implanted devices or leads. As described in detail below, in some embodiments a combination with a CRM apparatus includes adding pacing and/or defibrillation to the therapeutic actions included in the dynamic prescription of several embodiments of the present invention.

In further embodiments of the implantable impedance device and method for detecting and treating cardiovascular disease includes a cardiac rhythm management (CRM) module. In one embodiment, the cardiac rhythm management module includes a pacemaker. The term pacemaker includes antibradycardia and antitachycardia types. The term pacemaker also includes single chamber, dual chamber, and cardiac resynchronization therapy (CRT) types, the latter also called a biventricular pacemaker. In another embodiment, the cardiac rhythm management module includes a defibrillator. The term defibrillator, as used herein, shall be given its ordinary meaning and shall include atrial and ventricular defibrillators with or without combination with any of the pacemaker types listed above, or other devices. In another embodiment, the cardiac rhythm management module includes related devices that do not electrically depolarize all or some portion of the heart muscle to manage a cardiac rhythm or the synchrony of depolarization, but are used to perform some other function. For example, delivering electrical stimuli to cardiac muscle during the refractory period after depolarization may increase the strength of cardiac contraction, a phenomenon known as an ‘ionotropic’ effect.

It will also be known to those skilled in the art that pacing multichamber sites in appropriate sequence in addition to the atria, such as the right ventricle and the lateral wall of the left ventricle in combination, or the lateral wall of the left ventricle alone, has specific advantages for some patients with congestive heart failure due to enhanced synchrony of left ventricular contraction.

In another embodiment, the system is combined with or incorporated into a CRM system, with or without physiologic rate control, and with or without backup cardioversion/defibrillation therapy capabilities.

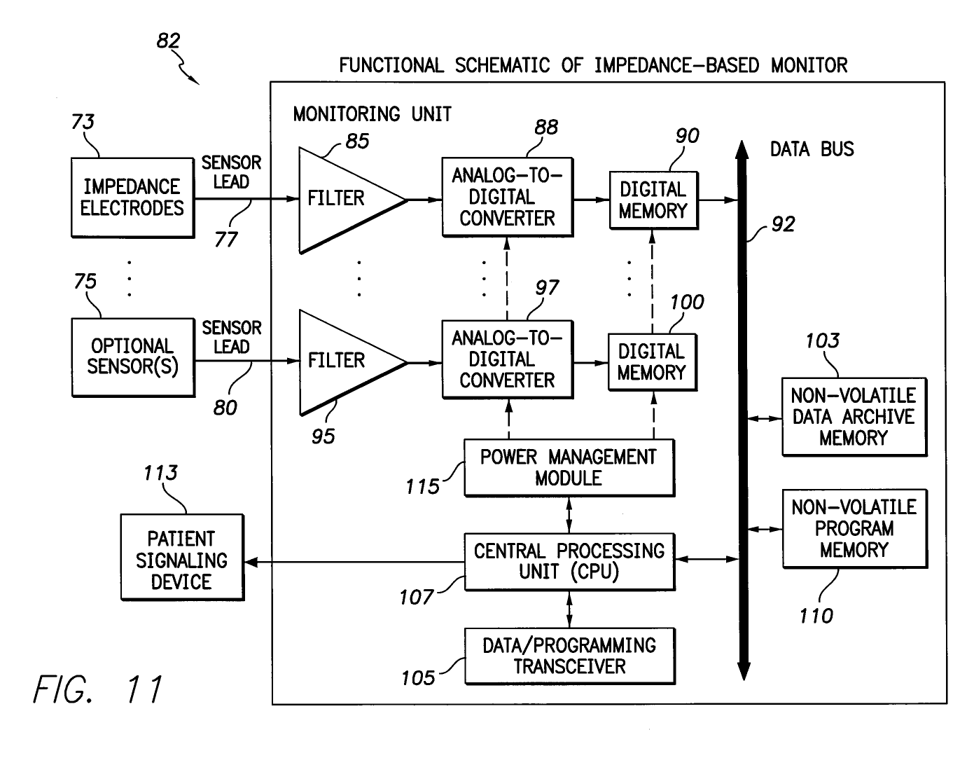

Referring to FIG. 10, in one embodiment the housing 7 includes a coil antenna 161 for communicating the one or more physiological signals from sensor package 15 to an external patient advisory module 6. In one embodiment, the external patient advisory module 6 includes a telemetry module 164 and antenna 162, a barometer 165 for measuring atmospheric pressure, and a signal processing/patient signaling device 166, such as described above with reference to FIG. 8.

In one embodiment of the invention, components of the impedance measurement apparatus for treating congestive heart failure are shared with the components of a CRM apparatus in such a way that, while sharing components, the two systems function essentially independently. In one embodiment, the implantable CRM apparatus generator has a housing that also serves as the housing for at least some components of the apparatus described in greater detail above. In a further embodiment, the power supply of the CRM apparatus, typically comprising a long lifetime battery and power management circuitry, also supplies power for one or more components of the apparatus for treating congestive heart failure. In yet another embodiment, the flexible lead or leads connecting the sensors, such as impedance measurement components, of the apparatus of FIG. 4, FIG. 5, and FIG. 7, to a shared housing/generator are also coupled to sensing and/or pacing electrodes of the CRM apparatus.

In one embodiment, one or more separate leads coupled to one or more impedance electrodes described above is also coupled to the CRM apparatus. In this embodiment, the CRM apparatus shares its generator housing with components of the implantable heart monitor apparatus described above, but the CRM apparatus leads are separate from the physiological sensor leads. In another embodiment, the pressure sensing lead may be combined with a pacing lead, as described for example by Pohndorf (U.S. Pat. No. 4,967,755) or Lubin (U.S. Pat. No. 5,324,326), herein incorporated by reference.

2. Integration of Impedance Signal Package and Pacing Lead

In one embodiment of the present invention, a system and method is provided for combining a CRM apparatus, implantable heart monitor, and patient communication device. The system provides the following functionality via a single pacing/sensing lead which in one embodiment includes only two conductors: (1) provides power to the impedance measurement module(s); (2) provides signaling for atrial pacing and sensing; (3) provides for programming of the non-impedance sensor package(s); and (4) provides measurement data from the physiological sensor package(s) to the monitor/defibrillator housing for immediate or delayed use by the patient, doctor or other caregiver via the patient signaling module. Additional sensor or pacing leads may be added.

In another embodiment, the measurement of pressure or other physiological parameters, such as thoracic impedance, may be multiplexed with the pacing signal (as described in greater detail below) so that pressure or impedance sensing and telemetry would occur between pacing signals, for example as taught by Barcel (U.S. Pat. No. 5,275,171) or Weijand et al. (U.S. Pat. No. 5,843,135), both incorporated by reference herein.

In one embodiment, a pacemaker is provided in which the electronics for producing the pacing pulse output and for sensing the ECG are integrated within a sensor package at the site of the pacing electrode, which is generally implanted within the heart. This allows the lead conductors to be substantially isolated from the pacing electrode, thereby providing increased immunity from induced currents when, for example, the patient is placed in the rapidly changing, strong magnetic fields of a magnetic resonance imaging machine. The lead may incorporate one or more sensors without requiring additional lead conductors.

In one embodiment, the system allows sensing signals to be processed within the heart, thereby eliminating the risk of picking up noise with lead conductors. Separate sensing and pacing electrodes may be provided, with no additional lead conductors. This allows the sensing and pacing electrodes to be individually optimized. Pacing electrodes are optimally small in area to minimize required voltage for pacing.

3. Upgrade from Stand-Alone to Combination System

The same sensor and lead 318 can be used either as part of a Stand-Alone system (such as a heart monitoring system, pressure monitoring and feedback system, HeartPOD™, POD, or apparatus for treating congestive heart failure, as described above) or as part of a combination system that includes a CRM or automated therapy system. This flexibility allows for the implantation of a Stand-Alone intracardiac module 320 that can be “upgraded” to include pacing and/or defibrillation therapy if the need arises without having to implant an additional lead. The combination system also allows the communication coil module 302 of the apparatus for treating congestive heart failure (such as that described above with reference to FIG. 7) to be removed and replaced with a CRM 306.

In one embodiment, the system is designed to operate in at least two different configurations, and in at least two modes of operation. A first mode is the “Stand-Alone Configuration.” A second mode is “the CRM Combination” (or “Combination Configuration”). One advantage of a multi-configuration system is that it allows the device to be implanted as a Stand-Alone system for CHF therapy and later to be upgraded for use with a CRM device if the patient's condition changes. In the Combination Configuration, in one embodiment, the sensor module 320 acts as a pace/sense electrode for the CRM device.

In one embodiment, various operational modes and parameters are programmed using an external programming device (not shown) that communicates with the implanted pacemaker transcutaneously using telemetry system 412, which decodes programming commands from a programmer and passes them to the programming circuitry 416. In one embodiment, physiological sensor signals, such as but not limited to thoracic impedance, pressure, temperature, or internal electrocardiogram signals, are passed from the communication circuitry 414 to the telemetry circuitry 412 for telemetry to the external patient advisory module, such as the patient advisory module illustrated and described above with reference to FIG. 7. In one embodiment, physiological sensor signals are also communicated from the communication circuitry 414 to the programming circuitry 416, where they are used to at least partially to control the operation of the pacemaker in response to the patient's condition.

a. CRM-Based Implant Therapy Units

In some embodiments, the non-electrical therapy may be used synergistically with cardiac electrical pacing or defibrillation in response to changes in physiological parameters in accordance with the present invention by, for example, AV delay optimization or any number of other methods, as are well known to one skilled in the art of cardiology and as described by Mann et al. in U.S. Pat. No. 6,970,742, herein incorporated by reference in its entirety. In one specific embodiment, the therapy delivery unit is configured deliver drugs in combination with cardioversion therapy in accordance with Advanced Cardiac Life Support (ACLS) protocols specified by the American Heart Association. The therapy delivery unit may be configured to provide pharmaceutical support for all or selected conditions covered by the ACLS protocol. In some embodiments, the selected conditions are individualized to the likeliest risks of any one patient. The patient advisory module may track the progression of the automated device through the ACLS protocols so that emergency personnel that are summoned during such an event can gain immediate access to the rhythms detected and drugs delivered.

Typically, in ACLS mode or “rescue mode”, the patient's condition is not amenable to a change in oral medication dose (see “Dynamic Prescription”). Thus, in one embodiment, this invention includes both the dynamic prescription with patient signaling, and automated therapy via electrical stimulation, drug infusion, or other therapy delivery unit. Drugs that may be so administered include but are not limited to natriuretic peptides (e.g., Natricor), diuretics (e.g., furosemide), and inotropes (e.g., epinephrine, norepinephrine, dopamine, dobutamine, milrinone). In one embodiment, rescue mode emergency drug infusion, defibrillation, or other therapy is performed automatically based at least in part on signals indicative of the patient's condition derived from the one or more sensors, such as an impedance sensor, of the invention. In another embodiment, rescue mode therapy is initiated by the present invention only after receiving doctor authorization to deliver the therapy. In one embodiment, doctor authorization is given by entering a password into the external patient signaling/communication module. This permits potentially dangerous emergency therapy to be delivered only after consultation with and authorization by a qualified healthcare professional.

### C. Telemetry

In one embodiment of the invention, one or more signals are communicated between the permanently implanted components of the system and a component of the system external to the patient's body. In one embodiment, signaling from the implanted to the external components is achieved by reflected impedance using radio frequency energy originating from the external device, and signaling from the external components to the internal components is achieved by frequency or amplitude shifting of radio frequency energy originating from the external device. Thus, in this embodiment, the current invention allows for telemetry of data from within the heart without transmitting radio frequency energy from the implanted device, advantageously resulting in significantly reduced power consumption compared to implants that perform telemetry by transmitting signals from within the body.

In another embodiment, signaling from the implanted to the external components is achieved through the metal housing of the implanted device using the method of Silvian (U.S. Pat. No. 6,301,504) incorporated by reference in its entirety.

In yet another embodiment, signaling from the implanted housing containing components of a CRM device is achieved via an antenna embedded within a dielectric around the periphery of the housing, as taught, for example, by Amundson et al. in U.S. Pat. No. 6,614,406, included herein by reference.

### D. Power

In one embodiment of the invention, the implanted apparatus is powered by a battery located within an implanted housing, similar to that of a cardiac pacemaker, as is well known in the art of cardiac pacing. In another embodiment, the implanted apparatus is powered by an external power source through inductive, acoustical or RF coupling.

### E. Digital Pacemaker Lead and Electrode

As described above, in several embodiments a cardiac rhythm management apparatus includes a pacemaker. In some embodiments, the cardiac rhythm management apparatus includes a “digital electrode.” In one embodiment, as used herein, a digital pacemaker shall be given its ordinary meaning and shall also mean a pacemaker in which digital signals, including energy pulses, are communicated between the proximal housing, or generator unit, and a distal module. Examples of digital electrodes that may be used in some embodiments of the invention are described in greater detail in U.S. Patent Publication No. 200510165456A1, herein incorporated by reference in its entirety. In one embodiment, the distal module comprises a digital electrode module, as described below. In another embodiment, the distal module comprises both a digital electrode and a sensor package or module. In one digital pacemaker embodiment, the digital signals include control signals to control the transfer of energy stored in the proximal housing to an energy storage device in the distal module. Energy pulses are transmitted from the proximal housing to the distal module, where the energy is stored in the distal module until delivery to the patient's heart. In another embodiment, the digital signals include sensor signals that are transmitted from the distal module to the proximal housing, from which they may be telemetered to an external device, such as a patient signaling device, as described in greater detail above. The distal module may comprise a sensor housing that includes electrodes, sensors, and electronic circuits. In other embodiments, as described in greater detail below, the distal electrode may include only a single electrode and electronic circuits.

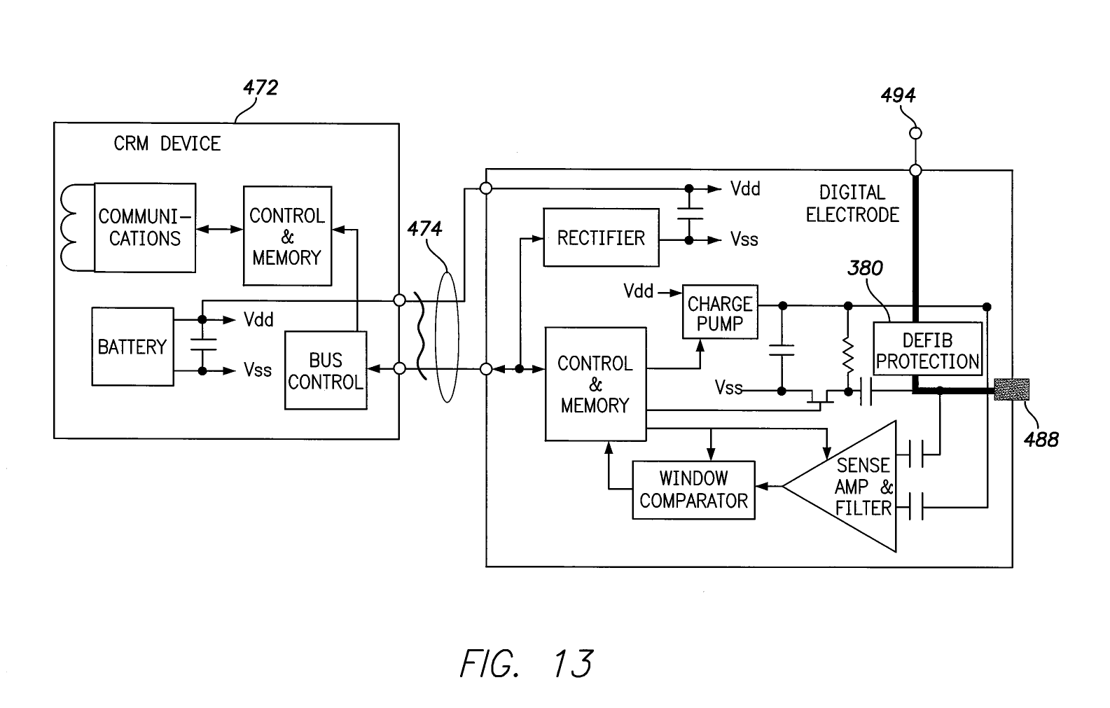

In one embodiment, depicted in FIG. 12, the electrode and sensor module 488 includes at least one electrode as described above. In one embodiment, the electrode and sensor module 488 includes an electrode for providing pacing stimuli to the heart, and a separate sensing electrode (not shown) to measure and/or sense the electrical activity of the heart. In another embodiment, the electrode and sensor module 488 includes at least one physiological sensor for measuring a physiological parameter of the heart. In one embodiment, the physiological sensor is a thoracic impedance sensor, pressure sensor, thermometer, ultrasonic sound emitter, ultrasonic sound receiver, IEGM sensor and/or any other sensor as described above, or as known to those of skill in the art. In one embodiment, the physiological parameter is a thoracic impedance indicative of the fluid volume within the lungs, a pressure indicative of the pressure within the left atrium of the heart, a temperature indicative of the patient's core temperature, an acoustic signal indicative of a volume of a chamber of the heart, or an electrical signal indicative of the pulsing and/or beating of the patient's heart.

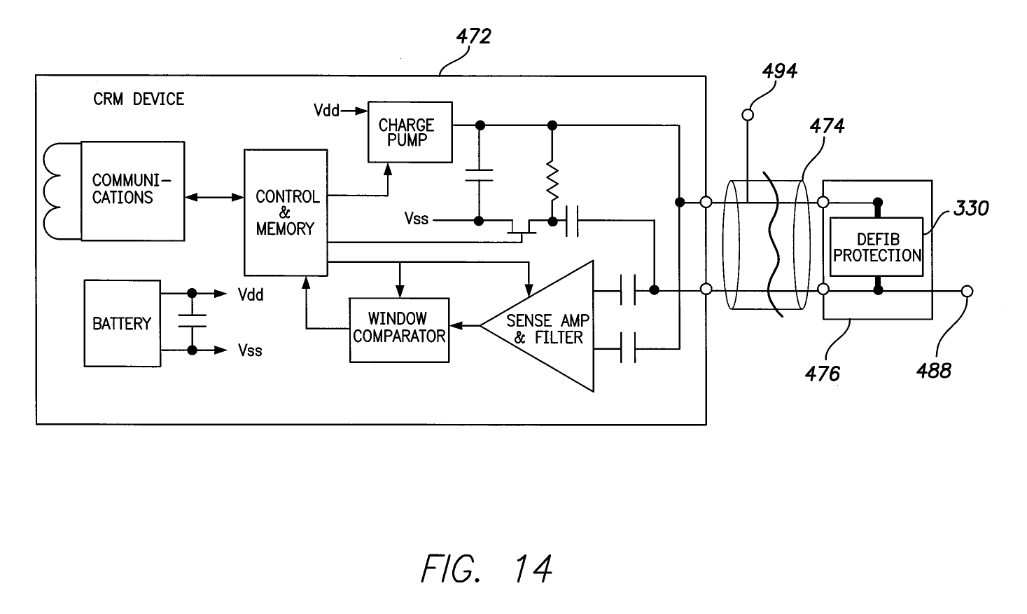

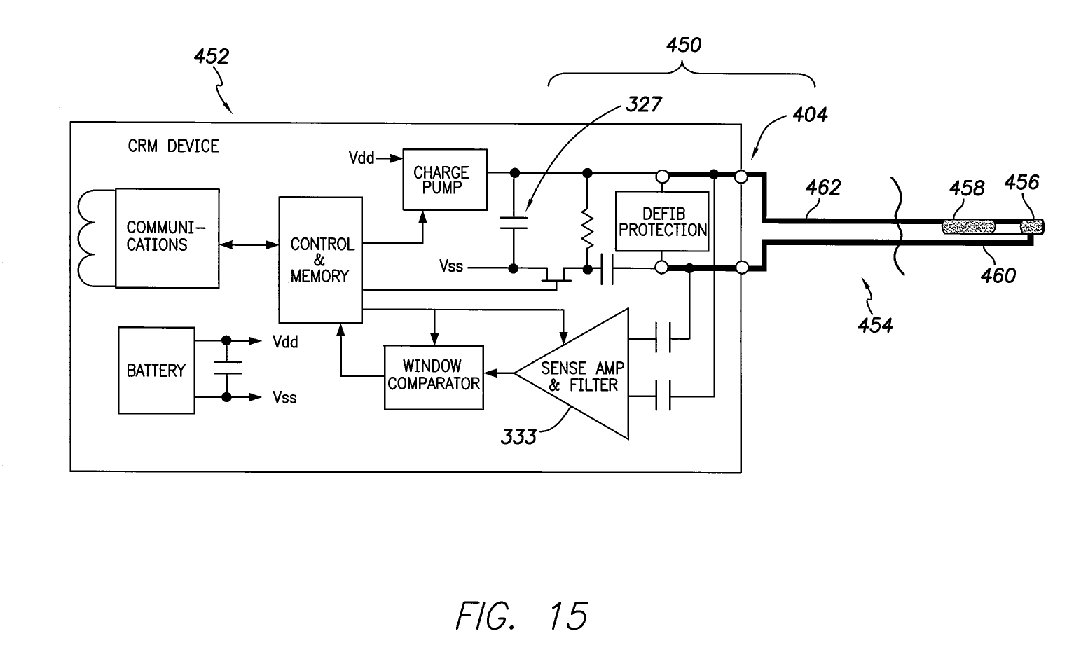

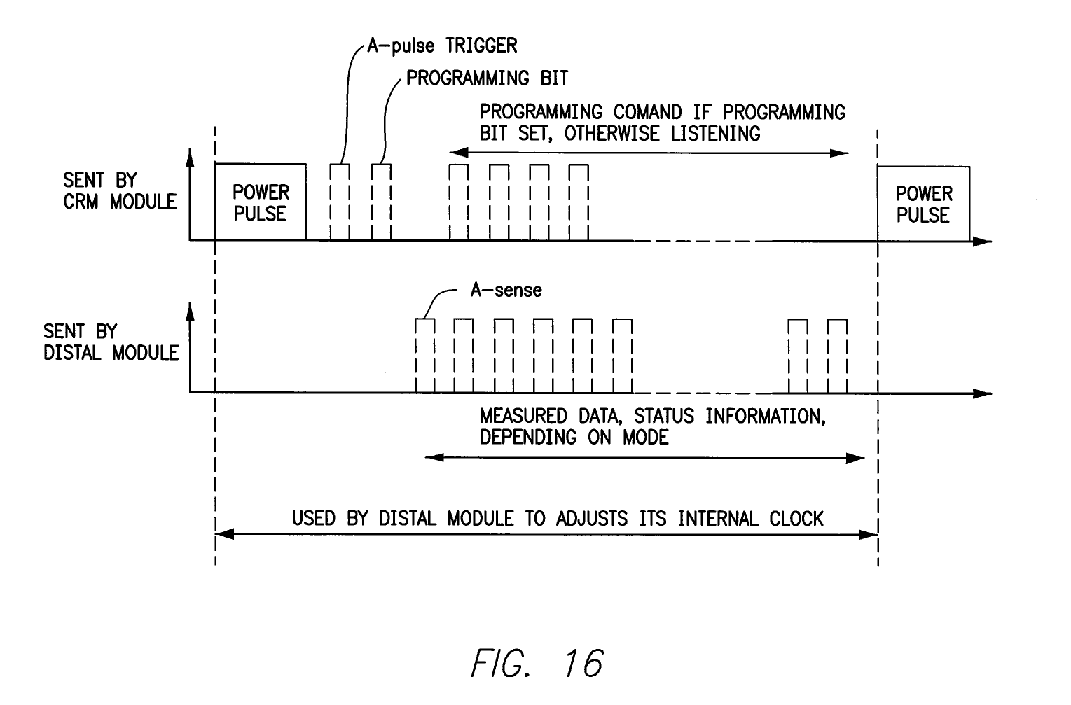

In several embodiments, the distal electrode module comprises the defibrillation protection circuitry 330, as shown for example in FIGS. 12 to 14. Referring now to FIG. 14, a CRM device is shown comprising a proximal housing 472, a lead 474, and a distal electrode module 476, in which the defibrillation protection is located in the distal electrode module. This has the advantage that the conduction path from the pacing electrode 488 to the indifferent electrode 494 (shown in bold lines) is very short, and is almost entirely within the distal electrode housing. This may be compared to the conduction path in the prior art CRM device (shown in bold lines in FIG. 15), which runs the length of the lead from the pacing electrode all the way back to the proximal housing. This aspect of the present invention reduces the effect of induced voltages due to magnetic resonance imaging.

### F. Digital Defibrillation

In another embodiment, a digital defibrillator includes an implantable heart monitor and a defibrillator, as described in greater detail above. In another embodiment, the implantable heart monitor includes any of the implantable heart monitors described above. The digital defibrillator provides power to the monitor, and it provides signaling for atrial and/or ventricular defibrillation, pacing, and sensing. In addition, the digital defibrillator includes a physiological sensor module that provides measurement data to a memory within a proximal housing. The digital defibrillator also allows the physiological sensor module to be programmed by an external device, such as a patient signaling module, as described in greater detail above.

### G. Digital Communication

Referring to FIG. 16, there is provided one illustration of a digital communication protocol over a two-conductor lead between a proximal housing and a distal module. In one embodiment the digital communication protocol is implemented in a digital pacemaker, and in another embodiment, the digital communication protocol is implemented in a digital defibrillator. Each power pulse-to-power pulse interval illustrated in FIG. 16 defines a frame of information over a particular time span. Each frame is further divided or segmented into a number of distinct sub-frame intervals. In one embodiment, each sub-frame interval is used to implement a defined function.

### III. SYSTEM OPERATION

### A. Signal Processing

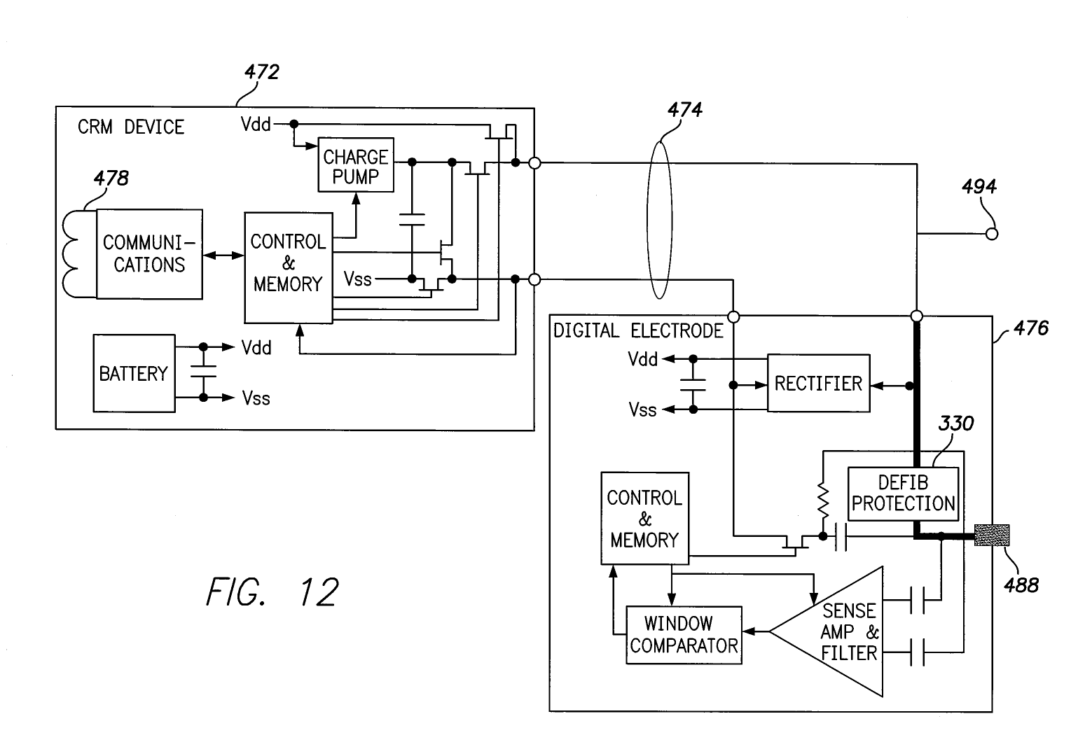

FIG. 11 is a schematic diagram of operational circuitry that in one embodiment is located inside the housing 7 (not shown) and is suitable for use in accordance with one embodiment of the present invention. The apparatus depicted in FIG. 11 includes digital processors, but the same concept could also be implemented with analog circuitry, as is well known to those of skill in the art.

As described above, in one embodiment, the system of the invention includes a thoracic impedance sensor 73 comprising at least one pair of impedance electrodes for measuring impedance. Moreover, the system may include one or more additional sensors 75 configured to monitor pressure at a location inside the left atrium, outside the left atrium, or a different physical parameter inside the left atrium or elsewhere. For each sensor 73, 75, a sensor lead 77, 80 conveys signals from the sensor 73, 75 to a monitoring unit 82 disposed inside the housing of the unit.

In one embodiment of the present invention, the digital data indicative of the thoracic impedance, as well as data corresponding to the other conditions detected by other sensors, where such are included, are transferred via the data bus 92 into a central processing unit 107, which processes the data based in part on algorithms and other data stored in non-volatile program memory 110. The central processing unit 107 then, based on the data and the results of the processing, sends an appropriate command to a patient signaling device 113, which sends a signal understandable by the patient and based upon which the patient may take appropriate action such as maintaining or changing the patient's drug regimen or contacting his or her physician.

Circuits or software for extracting relevant components from a thoracic impedance waveform are familiar to those skilled in the art. For example, a low pass filter element may be used to extract the long-term average, or “DC” component. In one embodiment, the outputs of overlapping low pass filters, one designed to include only frequencies lower than respiratory cycle frequencies, and the other designed to include respiratory but not cardiac cycle frequencies, are sampled at a fixed time in each cardiac cycle and subtracted to derive the respiratory component. The term of the long-term average is chosen to be long compared to the respiration rate but short compared to the rate of mean thoracic impedance change due to changes in a change in the patient's condition, so that slowly changing physiological information relevant to managing the patient's condition is not lost.

### B. Signal Communication

In several embodiments of the invention, the patient signaling device 113 comprises a mechanical vibrator housed inside the housing of the system. In one embodiment, the vibrator delivers a small, harmless, but readily noticeable electrical shock to the patient. In some embodiments, a low power transmitter configured to transmit information transcutaneously to a remote receiver, which could include a display screen or other means for communicating instructions to the patient.

In one embodiment, the signal processing and patient signaling components of the invention are combined into a patient advisory module, external to the patient's body. An additional advantage of this configuration is that it provides essentially unlimited storage for digital physiological data from the patient, as well as for information on medications and other relevant information to help the patient and physician manage congestive heart failure.

Yet a further advantage of the externalized patient signaling device component is that a much richer and easier to use interface with the patient is facilitated using a display screen and/or audio communication with the patient. In one embodiment, a reminder function is incorporated in the external device such that the patient is prompted to initiate measurement just prior to scheduled medications or other therapy. The patient is then advised of the appropriate doses of medications and/or other therapies based on the measurements and his physician's dynamic prescription.

In one embodiment, the patient advisory module is external and serves as a treatment and medications record. In this use, the patient will be asked to verify which of the prescribed medications were taken and which were, for whatever reason, were skipped, thus creating a record of compliance with the dynamic management program. This function will permit the physician to better manage the patient and, additionally, will improve patient compliance. Yet another advantage of the externalized patient advisory module is that it can be easily integrated with a cellular telephone or PDA/cell phone combination, allowing automated telemetry of alerts and/or physiological data to a remote health care provider such as the patient's physician, hospital, nursing clinic, or monitoring service.

Apparatus as described herein may also be useful in helping patients comply with their medication schedule. In that case, the patient advisory module could be programmed to signal the patient each time the patient is to take medication, e.g., four times daily. This might be done via an audio or vibratory signal as described above. In versions of the apparatus where the patient signaling device includes apparatus for transmitting messages to a hand held device, tabletop display, or another remote device, written or visual instructions could be provided.

Where the system includes apparatus for communicating information back to a base location, e.g., the hospital, doctor's office, or a pharmacy, the system in one embodiment, tracks the doses remaining in each prescription and to reorder automatically as the remaining supply of any particular drug becomes low.

In one embodiment of this invention, the external device communicates with a personal computer (PC) in the doctor's office either directly when the patient is present for an office visit, or via electronic communications, including, but not limited to, a telephone modem or the internet. During this communication, data is uploaded from the external device to the PC, including the records of physiological measurements, symptoms, and medication compliance, as well as information regarding the operation and calibration of the implanted device. Software on the PC displays the patient information, and the doctor enters a new dynamic prescription or edits the existing one. The PC then downloads the new or edited dynamic prescription to the external device.

### IV. EXAMPLES OF SYSTEM APPLICATION

### A. Example 1

Exemplary modes of operation for an embodiment of the system of the invention are described as follows. The following Example illustrates various embodiments of the present invention and is not intended in any way to limit the invention.

In one embodiment, the system is programmed to power up once per hour to measure the thoracic impedance and other conditions as dictated by the configuration of the particular system and any other sensors that might be present. Thoracic impedance measurements are taken at a 20-Hertz sampling rate for sixty seconds, yielding 1200 data values reflective of the amount of fluid in the lungs. The central processing unit then computes the mean thoracic impedance based on the stored values. Then, if the mean thoracic impedance is above a threshold value predetermined by the patient's physician, the central processing unit causes an appropriate communication to be sent to the patient via the patient signaling device.

Referring to FIG. 11, a set of coded communications to the patient can be devised by the treating physician and encoded into the device either at the time of implantation or after implantation by transcutaneous programming using data transmission into the non-volatile program memory 110 via the transceiver 105. For example, assume that the physician has determined that a particular patient's mean thoracic impedance can be controlled with drug therapy. This drug therapy might have been found to comprise a drug regimen including 5 milligrams (mg) of Lisinopril, 40 mg of Lasix, 20 milliequivalents (mEq) of potassium chloride, 0.25 mg of Digoxin, and 25 mg of Carvedilol, all taken once per day.

The patient is implanted with the device and the device is programmed as follows. The device includes a thoracic impedance sensor implanted in the thoracic cavity such that a portion of the lungs is between the sensor and the device housing. This thoracic impedance is a measure of fluid levels in the lungs which is correlated with, and thus indicative of, the left atrial pressure. The device's programming provides for four possible “alert levels” that are specified according to mean thoracic impedance detected by the sensor and computed in the central processing unit, and that the patient signaling device is a patient advisory module capable of displaying data and instructions to the patient.

At predetermined intervals, for example, hourly, daily, weekly, monthly, 3-4 times per day, or in response to a detected event, in response to a symptom, or in response to an instruction, the device measures the patient's mean thoracic impedance as described above, and determines the appropriate alert level for communication to the patient according to programming specified by the physician. For example, a mean thoracic impedance greater than normal thoracic impedance values could be indicative of some degree of over-medication and would correspond to alert level one. A thoracic impedance in the normal range would indicate optimal therapy and correspond to alert level two. A thoracic impedance that is slightly lower to moderately lower than normal thoracic impedance values would indicate mild under-treatment or mild worsening in the patient's condition to moderate under-treatment or moderate worsening in the patient's condition, respectively, and would correspond to alert level three. Finally, a mean thoracic impedance substantially lower than normal thoracic impedance values would indicate a severe worsening in the patient's condition, and would correspond to alert level four.

When the proper alert level is determined, the device sends an alert, such as a beep or vibrating pulse, to notify the patient that the device is about to communicate an alert level through the patient advisory module. Shortly thereafter, the alert level is displayed by the patient advisory module. Once the patient is informed of the alert level, the patient can continue or modify his own therapy with reference to a chart or other instructions prepared for him by the physician.

For example, for an alert level two, which signifies that the patient is within normal conditions, the doctor's instructions tell the patient to continue his or her therapy exactly as before. The signal for alert level two is given once every 24 hours, at a fixed time each day. This serves mainly to reassure the patient that the device is working and all is well with his therapy, and to encourage the patient to keep taking the medication on a regular schedule.

An alert level one likely indicates some degree of recent over-medication. The doctor's orders then notify the patient to reduce or omit certain parts of his therapy until the return of alert level two. For example, the doctor's instructions might tell the patient temporarily to stop taking Lasix, and to halve the dosage of Lisinopril to 2.5 mg per day. The coded signal is given to the patient once every twelve hours until the return of the alert level two condition.

Alert level three indicates a condition of mild worsening in the patient's condition. Accordingly, the doctor's instructions notify the patient to increase the diuretic components of his therapy until alert level two returned. For example, the patient might be instructed to add to his to his normal doses an additional 80 mg of Lasix, twice daily, and 30 mEq of potassium chloride, also twice daily. The level three alert signal would be given every four hours until the patient's condition returned to alert level two.

Alert level four indicates a serious deterioration in the patient's condition. In this case, the patient is instructed to contact his physician and to increase his doses of diuretics, add a vasodilator, and discontinue the beta-blocker. For example, the patient might be instructed to add to his therapy an additional 80 mg of Lasix, twice daily, an additional 30 mEq of potassium chloride, twice daily, 60 mg of Imdur, twice daily, and to stop taking the beta-blocker, Carvedilol. The signal corresponding to alert level four would be given every two hours, or until the physician was able to intervene directly.

### B. Example 2

In one embodiment, the system is configured as an externally powered implantable device with an impedance sensor system implanted in the thoracic cavity. A portion of the patient's lung is between the sensor and the device housing.

The temperature at the site of the sensor and an internal electrocardiogram (IEGM) are also detected by the sensor. A digital signal is communicated to an external telemetry device via an antenna coil implanted under the patient's skin and connected to the sensor by a flexible lead. The sensor is powered by radio frequency energy received by the implanted coil from an external coil connected to the external telemetry device. The external telemetry device forms part of an external patient advisory module, that also includes a battery power source, a signal processor, and a patient signaling device that consists of a personal data assistant (PDA) with a display screen and software for communicating with the patient.

The external patient advisory module is programmed to alert the patient at times determined by the physician, preferably at the times the patient is scheduled to take prescribed medications, typically one to three times per day. In one embodiment, the alert consists of an audible alarm and the appearance of a written message on the graphical interface of the patient-signaling device. The message instructs the patient to perform a “heart check,” that is to obtain physiological measurements from the implanted device. Instructions to the patient may include instructions to establish certain standard conditions, such as sitting quietly in a chair, prior to beginning the measurements. The patient is instructed to place the external telemetry/power coil over the implanted antenna coil, then to press a button to initiate the measurement sequence. Once the patient presses the button, the external device begins emits energy via the external coil to power and communicate with the implanted device. In one embodiment the external device emits an audible signal while communication is being established, then emits a second audible signal distinct from the first when communication has been established and while the measurement is taking place. Once the measurement is concluded, typically after 5 to 20 seconds, a third audible signal, distinct from the first two, is emitted to signal the patient that the measurement is complete.

In one embodiment, the external device will further instruct the patient, using its graphical interface, to enter additional information relevant to the patient's condition, such as weight, peripheral blood pressure, and symptoms. The signal processing apparatus of the external device then compares the measured physiological parameters from the implanted device, together with information entered by the patient, with ranges and limits corresponding to different therapeutic actions as predetermined by the physician and stored in the external device as a dynamic prescription, or DynamicRx™ The prescribed therapeutic action will then be communicated to the patient on the graphic display.

In one embodiment, the patient signaling apparatus will prompt the patient to confirm that each prescribed therapy has been performed. For example, if the therapy is taking a specific dose of oral medication, the patient will be prompted to press a button on the graphical interface when the medication has been taken. In one embodiment of the invention, this information is used to keep track of the number of pills remaining since the last time the patient's prescription was filled, so that the patient or caregiver can be reminded when it is time to refill the prescription.

As an example of a DynamicRx™ for a congestive heart failure patient, the level and rate of change of thoracic impedance may be used by the physician to determine the dosage of diuretic. If the thoracic impedance remains in the normal range for that patient, the patient signaling device would display the normal dosage of diuretic. As in Example 1 above, if the thoracic impedance is above the patient's normal range, the doctor may prescribe a reduction or withholding of diuretic, and that instruction would appear on the graphical interface. In another embodiment of a DynamicRx™ the patient may be instructed to take some other kind of action, such as calling the physician or caregiver, altering diet or fluid intake, or getting additional rest. Thus, the apparatus and methods of the present invention allow the physician to conditionally prescribe therapy for the patient, and to communicate the appropriate therapy to the patient in response to dynamic changes in the patient's medical condition.

In one embodiment, the physician enters the therapeutic plan for the patient, e.g., the DynamicRx™, on a personal computer and the DynamicRx™ is then loaded from the PC into the patient advisory module. In one embodiment, the patient advisory module is a PDA using the PALM OS® (Palm Computing, Inc.), or like, operating system and the DynamicRx™ is loaded from the physician's PC via the HOTSYNC® (Palm Computing, Inc.), or like, facility of PALM OS®. Loading of the DynamicRx™ from the physician's PC could be performed in the physician's office, or could be performed over a telephone modem or via a computer network, such as the Internet.

In one embodiment, DynamicRx™ software running on the PC contains treatment templates that assist the physician in creating a complete DynamicRx™, such that appropriate therapies/actions are provided for all possible values of the patient's physiological parameters.

In one embodiment of the present invention, the DynamicRx™ includes a patient instruction. In one embodiment, the patient instruction may includes directions or instructions to take medications, instructions to call 911, instructions to rest; or instructions to call a physician or medical care provider. In another embodiment of the present invention, one or more devices are provided to enable a physician or medical care provider to provide instruction to the patient. These devices include, but are not limited to, workstations, templates, PC-to-Palm hotsync operations, uploading processes, downloading processes, linking devices, wireless connections, networking, data cards, memory cards, and interface devices that permit the physician instruction to be loaded onto a patient's signal processor. In another embodiment, a user instruction is provided, where the user includes a patient, a physician, or a third party.

### C. Example 3

Heart failure patients implanted with the embodiments described in the above two examples may at the time of such implantation, or subsequently develop a medical indication for concurrent implantation of a CRM device. For example, required heart failure treatment with beta-blocking medication may slow the heart rate sufficiently to induce symptoms such as fatigue, or may prevent the heart rate from increasing appropriately with exertion, a condition known as chronotropic incompetence. These conditions are recognized indications for atrial pacing or atrial pacing with a rate responsive type of pacemaker. Normally this involves the placement of a pacemaker generator and an atrial pacing lead usually positioned in the right atrial appendage. In many cases, a dual chamber pacemaker is placed to synchronously pace the right atrium via one lead and the right ventricle via a second pacing lead. In other cases, such heart failure patients may have an abnormality of electrical conduction within the heart such as is known to occur with a condition called left-bundle branch block that causes dysynchronous left ventricular contraction thereby worsening heart failure. Implantation of a biventricular pacemaker has been shown to improve many of these patients. Because severe heart failure also carries an increased risk of sudden cardiac death due to a ventricular cardiac tachyarrhythmia, many of these patients are now being treated with implantable cardiac defibrillators (ICD's). In some cases combination rhythm management devices comprised of a biventricular pacemaker and an ICD are implanted.

In such cases where a CRM device is needed, it would be beneficial to the patient if the rhythm management device were integrated with the heart failure management devices described by Eigler et al., in U.S. Pat. No. 6,328,699 and U.S. Patent Application Publication Nos. 2003/0055344 and 2003/0055345, all of which are incorporated by reference in their entireties, to utilize the sensing lead yielding a thoracic impedance additionally as an atrial pacing lead. It would be further beneficial if the thoracic impedance sensing lead system described in Example 2 could be upgraded to combination heart failure management/CRM device by replacing the coil antenna with an appropriately integrated CRM generator without removing or changing the thoracic impedance sensing lead.

In one embodiment, the implanted heart failure device of Example 2 above is modified by replacing the implanted communications coil with an appropriately integrated CRM generator and additional pacing/ICD leads. The thoracic impedance sensing lead is connected as the atrial pacing lead to the generator. The generator has appropriate circuitry to power the sensing circuitry of the atrial lead. Thoracic impedance is read out by telemetry between the external PDA and the telemetry coil in the housing of the integrated rhythm management generator. If clinically appropriate, right and left ventricular pacing or defibrillation leads can be placed and connected to the generator. There are many potential benefits from such a combined rhythm and heart failure management system in addition to the clinical benefits from each individual system. Fewer leads need to be placed in the heart and a single venous insertion site can be used with the combined system. Atrial pacing from the intra-atrial septum has been show to inhibit paroxysmal atrial fibrillation, an arrhythmia common in heart failure patients. Patients can be titrated to higher or more appropriate beta-blocker dose levels with potentially increased survival benefits. Also, when thoracic impedance is within the desired normal range and thus the patient is not in acute heart failure, synchronous ventricular pacing can be inhibited to prolong battery life. It is understood by those skilled in the art, such as cardiologists and cardiac surgeons, that there may be additional clinical benefits bestowed by the combination of heart failure and rhythm management devices.

While this invention has been particularly shown and described with references to embodiments thereof, it will be understood by those skilled in the art that various changes in form and details may be made therein without departing from the scope of the invention. For all of the embodiments described above, the steps of the methods need not be performed sequentially.

What is claimed is:

1. A method for treating cardiovascular disease in a medical patient, the method comprising:
determining a thoracic impedance between a housing and an electrode, wherein the housing is located in the patient and wherein the electrode is located in the thoracic cavity of the patient;
determining the patient's spatial orientation;
communicating the thoracic impedance and spatial orientation to a signal processing apparatus;
operating the signal processing apparatus to generate a signal indicative of an appropriate therapeutic treatment to the patient,
wherein the signal is based at least in part on the thoracic impedance communicated to the signal processing apparatus and the patient's spatial orientation and
communicating the signal to the patient, wherein the signal comprises an instruction to the patient.

2. The method of claim 1, wherein the signal comprises at least two distinct instructions to the patient.

3. The method of claim 1, wherein the patient's spatial orientation is determined by querying the patient.

4. The method of claim 1, wherein the patient's spatial orientation is determined using a sensor configured to determine the patient's spatial orientation.

5. The method of claim 0, wherein the sensor comprises an accelerometer.

6. The method of claim 0, wherein the sensor comprises a multiaxis tiltometer.

7. The method of claim 1, further comprising:
delivering at least one electrical pulse from the electrode; and
measuring a voltage between the electrode and the housing in response to the electrical pulse.

8. A method for treating cardiovascular disease in a medical patient, the method comprising:
signaling the patient to assume a spatial orientation;
determining a thoracic impedance between a housing and an electrode, wherein the housing is located in the patient and wherein the electrode is located in the thoracic cavity of the patient;
communicating the thoracic impedance and spatial orientation to a signal processing apparatus;
operating the signal processing apparatus to generate a signal indicative of an appropriate therapeutic treatment to the patient,
wherein the signal is based at least in part on the thoracic impedance communicated to the signal processing apparatus and the patient's spatial orientation; and
communicating the signal to the patient, wherein the signal comprises an instruction to the patient.

9. The method of claim 8, further comprising:
delivering at least one electrical pulse from the electrode; and
measuring a voltage between the electrode and the housing in response to the electrical pulse.

10. The method of claim 8, wherein the signal comprises at least two distinct instructions to the patient.

11. The method of claim 8, wherein the patient is signaled to perform at least one of the following: adopt a prone position, sit and stand.

12. A system for treating cardiovascular disease in a medical patient, comprising
an implantable device,
wherein the implantable device comprises an electrode, a housing and an impedance measurement module, the electrode configured to be positioned within the thoracic cavity and configured to deliver at least one electrical pulse, the housing configured to be positioned in the patient, the impedance measurement module configured to measure the impedance between the electrode and the housing;
a signal processing module configured to generate a signal indicative of an appropriate therapeutic treatment to the patient based on the impedance measured by the impedance measurement module; and
a patient advisory module configured to receive the signal generated by the signal processing module and to communicate the signal to the patient, wherein the communicated signal comprises an instruction.

13. The system of claim 12, wherein the signal comprises at least two distinct instructions to the patient.

14. The system of claim 12, wherein the implantable device further comprises a sensor configured to determine the patient's spatial orientation.

15. The system of claim 12, wherein the sensor comprises an accelerometer.

16. The system of claim 12, wherein the sensor comprises a multiaxis tiltometer.

17. The system of claim 12, wherein the instruction advises the patient to take medication.

18. The system of claim 12, wherein the instruction advises the patient to see a physician.

19. The system of claim 12, wherein the instruction advises the patient that the patient's health status is unchanged and no action is needed.

20. The system of claim 12, wherein the implantable device further comprises a pressure sensor configured to measure left atrial pressure.
